(19) United States
(12) Patent Application Publication
(10) Pub. No.: US 2009/0221069 A1
(43) Pub. Date: Sep. 3, 2009

(54) METHOD FOR AUGMENTING THE ABILITY OF T-CELLS AND OTHER CELLS FOR FIGHTING DISEASE AND INVADE DISEASED ORGANS, FOR ELEVATING CD3 ZETA AND TNF-ALPHA EXPRESSION IN T-CELLS, AND MIXING T-CELL BOOSTING AND KIT PARTICULARLY USEFUL IN SUCH METHOD

(76) Inventor: Mia Levite, Savyon (IL)

Correspondence Address: BROWDY AND NEIMARK, P.L.L.C.;624 NINTH STREET, NW, SUITE 300, WASHINGTON, DC, 20001-5303, US

(21) Appl. No.: 11/911,934

(22) PCT Filed: Apr. 20, 2006
(86) PCT No.: PCT/IL06/00482

§ 371 Date: Apr. 24, 2008

Related U.S. Application Data
(60) Provisional application No. 60/671,546, filed on Apr. 18, 2005.

(51) Int. Cl. C12N 5/08; C12M 3/00

(52) U.S. Cl. 435/372.3; 435/366; 435/305.3

(57) ABSTRACT

A method and device for augmenting the ability of T-cells, NK cells and other body cells, of a patient for fighting disease and invading diseased tissue, and for elevating the level of CD3 zeta expression in T cells for better functioning of these T-cells and their augmented capability to eliminate cancer and infectious organisms, by exposing, for a predetermined exposure period, the first substance of T-cell or other cells to a second substance having a T-cell-boosting capability, such as, but not limited to CD3 zeta elevating capability, increased CXCR4, CD147, 67-kDa laminin receptor and/or any other key receptor in T-cells capability, increased tissue invasion capability, TNFa elevating capability, to augment the ability of the separated cells to fight the disease; and following the predetermined exposure period, reintroducing the first substance of T-cells back into the blood of the subject. The first substance is exposed to the second substance by mixing the two substances in a mixing device providing hermetically-sealed conditions effective to maintain sterility in both substances during the mixing and to maintain tissue culture conditions required for the cells viability thereof and for the required exposure period.

### FIELD AND BACKGROUND OF THE INVENTION

### T-Cells are Major Components of our Immune System and Crucial for Fighting Diseases

T-cells are major components of the immune system. Alike an efficient police, T-cells move and patrol in the body all the time, and can enter every organ of the body. T-cells are crucial for fighting foreign invaders like viruses and bacteria, and for destruction of cancer cells. Therefore, potent T-cell function is crucial for health. Abnormal, insufficient or absent T-cell function can lead to numerous diseases, and treatment of such diseases must take this into consideration.

T-lymphocytes originate from lymphocytic-committed stem cells of the embryo. T-cells include, but are not limited to CD4+ T cells [also known as T helper (Th1, Th2 and Th3 cells) and T inducer cells], CD8+ cytotoxic T-cells, and CD4+ CD25+ T suppressor/regulator cells (previously known as cytotoxic/suppressor T cells), which, when activated, have the capacity to lyse target cells and suppress CD4+ mediated effects. Other blood cells with important immune surveillance role are the natural killer cells. Interestingly, some key receptors in NK cells are also down regulated in cancer cells, (alike CD3 zeta in T-cells).

Other blood cells with different functions include, but are not limited to erythrocytes and platelets. Also present in blood are various types of blood cell progenitors, or hematopoietic cells, such as CD34+ hematopoietic stem cells.

Insufficient T-cell surveillance can lead to various pathologies, among them:

1. Growth and spread of cancer of various types, impairing key functions, potentially causing death of the affected individual;

2. Unchecked growth and spread of viruses, bacteria and other infectious organisms in the body, causing diseases and impairment of key functions, and even death of the organism.

Thus, “boosting” of T-cell function when it is lacking or insufficient, is desirable in various pathological conditions among them cancer (of various types), infectious diseases of various types (caused by viruses, bacteria, parasites, prion, proteins, etc.), immunodeficiency diseases of various types, after bone marrow transplantation (BMT), and even in old age, where there is a decrease in immune function in general, and lymphocyte number and function in particular, leading to increased susceptibility to diseases.

Cancer Patients have Anti-Tumor T-Cells in their Circulation and in Tumor-Bearing Organs, but these Fail to Provide Significant Protection Against Cancer Growth and Spread.

The central role played by T-cell subsets in the control of tumor growth progression has been widely accepted. Nevertheless, it remains unclear why T cells are unable to exercise this control successfully in most cases of malignancy, and tumor escape from host immune system surveillance remains one of the unsolved problems of modern immunology. For years, the scientific community has been reluctant to accept evidence that tumors are able to manipulate and subvert the host immune system. Recently, attitudes have changed, largely due to the realization that therapeutic cancer vaccines have not been as effective as expected in inducing immunologic and clinical responses, even when highly sophisticated vaccination strategies are used in “immunologically responsive” cancers, such as melanoma. Thus, despite high hopes, active vaccination has been a disappointment, so far. Apparently, tumors at their advanced stages, employ as yet poorly understood mechanisms to escape or evade immune surveillance. On the other hand, passive or adoptive vaccination using either anti-cancer antibodies or tumor-specific effector lymphocytes such as T- and natural killer (NK) cells has proven efficacious in clinical trials for certain antigenic/immunogenic tumors.

Modern technologies, including the use of tetramers or ELISpot assays, confirm that tumor-specific T cells are present in the circulation of patients with cancer, and often also in tumor-bearing organs, where they are called tumor-infiltrating lymphocytes (TIL's). It could be, therefore, expected that these T cells are able to effectively eliminate the tumor or arrest its metastases. Because they often do not, the old dilemma of why T cells in tumor-bearing hosts are unable to prevent or interfere with tumor growth, although they apparently can control invading pathogens, remains one of the most challenging aspects of tumor immunology today. One possible answer might be that tumor-specific T lymphocytes, although present, are not functional in the tumor microenvironment, and that this dysfunction is caused by the tumor, facilitating its escape from immune surveillance. This hypothesis is based on extensive evidence demonstrating dysfunction of TILs and of circulating T cells in patients with various malignancies. While the mechanisms involved in this dysfunction are complex, its manifestations translate into ineffective antitumor responses.

Thus, it would be advantageous to be able to compensate and restore function in a patient's T-cells, and provide them with “rejuvenated” abilities to fight the cancer.

### Tumor Specific T-Cells Used in Various Immunotherapy Protocols, are Often Limited Due to the Small Number of T-Cells Able to Invade Tumor-Bearing Organs.

Most importantly, various strategies of T-cell mediated immunotherapy of cancer, based on tumor-specific T-cells, as well as various vaccination methodologies in cancers, aimed to augment the elimination of cancer by tumor-specific T-cells, are often limited by the small number of T-cells able to invade tumor-bearing organs. Thus, reintroducing autologous tumor specific T-cells into the circulation of a cancer patient, often fails to have the desired effect, since insufficient number of T-cells in fact managed to leave the circulation and invade the tumor bearing organ (e.g. e.g. bone marrow, breast, prostate and others).

Thus, boosting invasion and/or homing of T-cells into solid tumors has become an important clinical challenge in immunotherapy of cancer. It is therefore clear why practically all T-cell immunotherapy protocols in cancer could benefit substantially from methodologies that can enhance invasion of T-cells into tumor-bearing tissues, and that could boost T-cell activity.

Impaired CD3 Zeta Expression in T-Cells of Patients with Cancer, Some Chronic Infections and Autoimmune Diseases.

The CD3 zeta chain: The TCR-associated zeta chain is a 16-kDa molecule consisting of a very short extracellular domain, a transmembrane region, and a long cytoplasmic tail, which contains three immunoreceptor tyrosine-based activation motifs (ITAMs). It usually exists as a homodimer, so that there are six phosphorylation sites or ITAMs on the two zeta chains in the TCR complex. In some T cells, zeta combines with the g or FcRc chain, forming a heterodimer with fewer phosphorylation sites. CD3 zeta functions as a transmembrane signaling molecule in T cells. Following TCR ligation, Src family tyrosine kinases, p561ck and p59fyn phosphorylate CD3 zeta, and trigger recruitment and activation of the Syk family kinases ZAP-70 and Syk, leading to NFkappaB activation downstream and its translocation to the nucleus. Expression of the CD3zeta chain is, therefore, essential for activation of T cells! (for a review of CD3zeta and cancer, see Whiteside Cancer Immunol Immunother 2004; 53:865-78).

TCR is a complex of several molecules, which cooperate in the process of recognition and binding of the peptide presented by the MHC on the antigen-presenting cell (APC). The formation of an immunologic synapse between the correctly assembled TCRs on the cell surface and the APC presenting a cognate MHC-peptide complex triggers the receptors. A productive TCR signal induces ordered successive phosphorylation of all six ITAMs on the CD3f homodimer. The process of T-cell triggering is self-limited, and down-regulation of triggered TCRs normally involves a loss of f protein. The mechanisms responsible for this loss are not clear, although internalization and lysosomal degradation of TCR chains, including the CD3 zeta chain, have been observed in experiments involving human T-cell clones specific for tetanus toxoid peptides. The T cells then proceed to replace the internalized receptors on the cell surface and to interact again with the immunogenic peptide. However, lysosomal degradation may not be the only mechanism of cellular degradation of CD3zeta, as discussed hereinbelow. Nevertheless, it is important to note that chronic antigenic stimulation via TCRs could lead to prolonged or even permanent downregulation of CD3zeta expression and to partial or complete T-cell anergy. Low or absent zeta-chain expression in circulating T cells is not confined to patients with cancer; it has also been documented in chronic infections such as leprosy and AIDS, as well as autoimmune diseases associated with circulating immune complexes, such as SLE.

Because of the central role of CD3zeta in cellular signaling, it's decreased or absent CD3zeta expression in lymphocytes translates into aberrant or inefficient signaling, resulting in a partial or complete loss of immune functions. For this reason, CD3zeta expression in lymphocytes has been a focus of attention, and several different techniques have been used to evaluate it in cancer and other diseases.

Evidence for Decreased CD3 Zeta-Chain Expression in T Cells of Patients with Cancer:

Recently, it has become evident that there is a markedly deficient expression CD3 zeta-chain in T cells of patients with various types of cancer, among them ovarian cancer (Lai P, et al Clin Cancer Res 1996; 2:161-173); melanoma (Zea A H, et al Clin Cancer Res 1995; 1:1327-1335); prostate cancer (Healy C G et al Cytometry 1998; 32:109-119); breast cancer; oral cancer (Reichert T E, et al Cancer Res 1998; 58:5344-5347); renal cancer (Finke J H, et al Cancer Res 1993; 53:5613-5616), head and neck cancer (Kuss I, et al Clin Cancer Res 1999; 5:329-334; and Cancer Immunol Immunother 2004; 53: 865-878), and colorectal carcinoma (Nakagomi H, et al Cancer Res 1993; 53:5610-5612; and Matsuda M, et al Int J Cancer 1995; 61:765-772). By and large, the lowest f expression was seen in tumor infiltrating lymphocytes (TILs); tumor associated lymphocytes (TALs); lymphocytes isolated from tumor-involved lymph nodes and peripheral blood T-cells obtained from patients with advanced metastatic disease.

Thus, it would be highly advantageous to have novel methods and devices for enhancing the CD3 zeta levels of T-cells in cancer and other, CD3 zeta-related diseases. To date, elevating CD3zeta in T-cells of cancer patients remain, a highly important yet unmet clinical goal.

Prognostic importance of the level of CD3 zeta chain expression in T-cells of patients with cancer: Several studies in recent years in cancer patients indicated that the higher CD3 zeta expression, the better the survival (for a review of CD3zeta and cancer, see Whiteside Cancer Immunol Immunother 2004; 53:865-78).

Due to the key role CD3 zeta plays in TCR signaling, it might be expected that the biological consequences of its low/absent expression are considerable, resulting in depressed anti-tumor immunity, poorer prognosis, and shorter overall survival. To address this issue, in a retrospective study (Reichert T E et al, Cancer Res 1998; 58:5344-5347) CD3 zeta chain expression was measured in TILs present in biopsy samples of oral carcinoma obtained from 138 patients who underwent curative surgery and for whom a follow-up of >5 years was available. Absent or low expression of CD3 zeta in TILs was detected in 32% of tumors and was significantly associated with a high tumor stage (T3 or T4) as well as nodal involvement. In patients with oral carcinoma and advanced disease, normal expression of CD3 zeta in TILs was predictive of significantly better 5-year survival independently of other established prognostic parameters. The same study showed, for the first time, that expression of CD3 zeta chain was identified as an independent prognostic marker in oral carcinoma, in this study (Reichert, et al, Cancer Res 1998; 58:5344-5347). In a more recently completed study, which utilized the same biopsy material, the same authors reported that the number of tumor-infiltrating dendritic cells was also a highly significant prognostic factor in patients with oral carcinoma. The absence or paucity of dendritic cells was strongly associated with abnormalities of f-chain expression in TILs (Reichert T E, et al Cancer 2000; 91:2136-2147). Low density of DCs and low or absent expression of CD3 zeta-chain in TILs correlated with each other and predicted the poorest survival and the greatest risk in this cohort of patients with oral carcinoma. These findings showed that a correlation existed between CD3 zeta chain expression in T cells accumulating at the tumor site and tumor progression, and suggested that f expression in TILs might serve as a biomarker of survival.

### The Expression Level of CD3 Zeta in T-Cells of Cancer Patients in Monitoring of Clinical Trials:

Among 19 patients with ovarian carcinoma receiving intraperitoneal IL-2, 9 clinical responders to therapy had normal CD3 zeta expression in circulating T cells prior to therapy, while in 10 non responders to IL-2, CD3 zeta expression was significantly decreased (Kuss I, et al Cancer Biother Radiopharmaceuticals 2002; 17:631-640). This observation reinforces the initial impressions that in patients with cancer, CD3 zeta may be a marker of immune competence in individuals most likely to respond favorably to biotherapy.

### Current Cancer Therapies can be Ineffective and Detrimental:

Most advanced disseminated cancers are by and large un-curable by the treatment modalities available today. Surgery, chemotherapy, and irradiation, have three significant drawbacks:

1. They often are unable to reduce/remove/eliminate the cancer.
2. Even in cases where these anti-cancer strategies are effective by reducing the tumor mass, they usually can not eliminate residual disease and single cancer cells that escape and become “sequestered” within tissues. Thus, in many cases, it is only a matter of time till the cancer re-occurs (i.e a relapse).
3. These anti-cancer strategies harm healthy cells and organs and weaken the immune system. In doing so they weaken dangerously the ability of patient's T-cells and natural killer (NK) cells to detect and eliminate the cancer cells wherever they are by T-cell mediated “targeted elimination”.
Neurotransmitters, at Physiological Concentrations, can by Themselves Activate Various Key T-Cell Functions, Augment Migration and Homing into Tissues, and Upregulate the Expression of CD3zeta of Other Crucial Receptors:

In the last few years Dr. M. Levite et al found that neurotransmitters by themselves (i.e. in the complete absence of any other molecules), at physiological concentrations can bind to their specific receptors in T-cells and activate (i.e. trigger/induce) various key T-cell features and functions (see, for example, M. Levite, et al. J. Immunol (1998) 160, 993-1000; M. Levite, et al. Proc. Natl. Acad. Sci. USA (1998) 95, 12544-12549; M. Levite, et al. J. Exp. Med. (2000) 191, 1167-1176; Besser, et al J Neuroimmunol. 2005 December; 169(1-2):161-71; M. Levite, et al. Eur. J. Immunol. (2001) 12, 3504-12; Chen, et al. Nature Medicine, (2002) December; 8(12):1421-1426; Ganor, et al. J Immunol, (2003) 170: 4362-4372; M. Levite New York Acad Sci. (2000) 917, 307-21; M. Levite. Trends in Immunology (2001) 22 (1); M. Levite and Y. Chowers Ann Oncol. (2001) 12 Suppl 2:S, 19-25; Mia Levite; Book chapter: “Neurotransmitters talk to T-cells in a direct, powerful and contextual manner affecting key immune functions” pp 263-288, in: ‘Immunoendocrinology in Health and Disease’, 2005, Editors: Vincent Geenen, George Chrousos Publishers; Marcel Dekker, New York). Thus, T cells, like neuronal cells (and others), express cognate receptors to a few neurotransmitters. These studies revealed that several key neurotransmitters, among them glutamate and dopamine, and various neuropeptides, among them Somatostatin, Neuropeptide Y, GnRH-I and GnRH-II, Substance P and Calcitonin-gene-related-peptide, can by themselves and in relatively very low concentrations (nM) potently trigger a kaleidoscope of T-cell functions, including:

1) Affecting the gating of voltage-gated potassium channels in T-cells and inducing rapid changes in the T-cell membrane potential (second-minute scale). Of note, voltage-gated potassium channels have shown in recent years to be crucial for initiating TCR-mediated antigenic stimulation and subsequent T-cell functions (e.g. Ref 12);
2) Activating integrin-mediated functions (e.g. adhesion to fibronectin and laminin).
3) Production and secretion of key cytokines, e.g. IFNg, TNFa, IL-2, IL-4 and IL-10. Moreover, the profile of neurotransmitter-induced cytokine secretion can be very different than that induced by a ‘classical’ T-cell receptor (TCR) stimulation, induced by an antigen, mitogen, etc.
4) Augmenting T-cells chemotactic migration in vitro towards key chemokines (e.g. SDF-1 and MIP-1b in-vitro.
5) Augmenting T-cell in vivo homing and invasion into specific organs (e.g. spleen, bone marrow and kidney).
6) Modulating the in vivo encephalitogenic and inflammatory behavior of T-cells (unpublished);
7) Triggering de novo expression of specific genes, some of which code for key T-cell receptors and enzymes.
8) Up-regulating the surface expression levels of CD3 zeta in T-cells a very significant and consistent manner (see Examples section hereinbelow).
9) Up-regulating the surface expression levels of the chemokine receptor CXCR4 in T receptors.
10) Up-regulating the surface expression levels of the metalloproteinase-induced CD147.
11) Up-regulating the surface expression levels of the 67-kDa laminin receptor, required for migration and homing of T-cells into tissues, for binding laminin, for retaining memory in inflamed sites and other key function.
12) Up-regulating the surface expression levels of other T-cell receptors.
13) Augmenting the ability of human tumor specific T-cells to reject human cancer in-vivo, (as shown in SCID mice).

All the above T-cell functions triggered by given neurotransmitters through their cognate receptors on T-cells, can be highly relevant to anti-tumor T-cell activity, and to anti-viral and anti-bacterial activity. Specifically, Dr. Levite et al obtained preliminary evidence that these newly uncovered functional interactions between T-cells and selected neurotransmitters and neuropeptides, primarily GnRH-II, dopamine, and glutamate augmented significantly the overall in vivo activity of human tumor-specific T-cells against human cancer growing in SCID mice, resulting in a significant augmentation of in vivo cancer rejection.

Increasing adhesion of T cells and activity of chemokine receptors are crucial for the ability of T cells to extravasate, traffic and home to their target, and neurotransmitters were found to increase such adhesion and migration.

It is known that certain types of blood cells, and particularly T-cells, have the ability of fighting certain types of cancer and infectious diseases, and that this ability can be augmented by exposing such cells to certain substances having a cell enhancing capability; for examples, see International Patent Application No. PCT/IL02/00870 published May 8, 2003 as International Publication No. WO 03/037247, International Patent Application No. PCT/IL02/01014 published Jun. 26, 2003 as International Publication No. WO 03/051272, and U.S. patent application Ser. No. 10/809,452 published Sep. 29, 2005 as US Publication No. 2005-0214217, the contents of which are incorporated herein by reference.

International Patent Application No. PCT/IL02/00870, to Levite, teaches activating or inducing de novo T-cell function such as, for example, β integrin binding, cytokine secretion and membrane depolarization using physiological concentrations of Dopamine, acting directly and by itself, on human and murine T cells via well characterized Dopamine receptors. Accordingly, dopamine- or dopamine analog-treated T-cells can be used for inhibition and prevention of tumor growth and dissemination, the treatment of bacterial, viral, fungal infectious and parasitic diseases, containment of auto-immune and other injurious inflammatory processes.

International Patent Application No. PCT/IL02/01014 discloses, for the first time, the direct action of Gonadotropin Releasing Hormone (GnRH I and GnRH II) on well characterized T-cell GnRH receptors, activating or inducing de novo numerous important T cell functions, such as, for example, induction of gene expression, most significantly of the 67 kD non-integrin laminin receptor, adhesion to laminin, chemotaxis and T-cell extravasation. Also taught are methods for the using such T-cell activated by GnRH and specific GnRH receptor functional analogs for the treatment of bacterial, viral, fungal infectious and parasitic diseases, containment of auto-immune and other injurious inflammatory processes, inhibition and prevention of tumor growth and dissemination.

US Publication No. 2005-0214217, to Levite, discloses, for the first time, the direct activating or inducing de novo of T-cell activity by the action of Glutamate and Glutamate functional analogs. Particularly, methods and compositions for using such glutamate-treated T-cells for the treatment of viral and other infectious diseases, anti-tumor immune surveillance prevention and treatment of neurological disease, psychopathology, neuronal damage in CNS disease, infection and injury. Also taught are methods to suppress T-cell activity by certain glutamate analogs for treatment of T-cell mediated auto-immune, T-cell mediated injurious inflammatory processes, T-cell cancers and prevention of T-cell mediated host rejection of engrafted tissue.

The current described method calls for ex-vivo strengthening numerous capabilities both of the freshly-isolated T-cells and the pre-processed cultured T-cells.

Manipulation of immune cells for therapy of brain related disorders has been proposed by Wank (Intern Pat. Publications WO9950393A2 and WO9950393A3 to Wank, R). Wank describes the in-vitro activation of peripheral blood monocytes (PBMC), or phagocytes, for the treatment of a variety of brain-related disorders, including psychoses, schizophrenia, autism, Down's syndrome, disturbances of cerebral development and brain injury, based on the observation of inadequate immune responses in these conditions. In a report documenting adoptive immunotherapy of patients suffering from bipolar disorder, schizophrenia or autism, Wank describes the in-vitro activation, and reintroduction of the patients' own T-cells, in order to combat “chronically infected”, understimulated lymphocytes thought associated with these disorders. In this form of therapy, the T-cells are not stimulated directly, rather via monoclonal antibodies against the CD3 polypeptide complex, and IL-2 added to the T-cells in cell culture dishes. The patients were required to endure numerous weekly treatments (up to 104 weeks in one patient), and although improvement in some symptoms was noted, additional therapies were continued during and after these trials of adoptive immunotherapy. No devices for mixing neurotransmitters or other substances with the T-cells were disclosed.

Ex-vivo stimulation of T-cells and other cells has also been disclosed for other applications. U.S. Pat. No. 5,866,115 to Kanz et al teaches a method and kit for preparing dendritic cells for re-introduction into a patient, by long-term exposure of the cells to IL3, IL6, stem cell factor, EPO, and optionally IL4 and GM-CSF. No mixing of cells with neurotransmitters are taught, nor are any devices for sterilely and accurately mixing the cells and boosting substances mentioned or taught.

Similarly, Ridihalgh (U.S. Pat. No. 6,713,054) teaches ex-vivo expansion and treatment of lymph node cells from Chronic Fatigue Syndrome patients, using IL-2 and anti-CD3 monoclonal antibodies, followed by reintroduction of the treated, expanded cells to the patient. However, the treatment disclosed is extremely long-term, and is based mostly on selection of subpopulations of the lymph cells. No boosting with neurotransmitters, or devices or kits for short-term T-cell boosting are disclosed. Further, Yang (US Patent Application No. 20060057121) teaches the ex-vivo activation and expansion of blood cells for administration to myelosupressed patients, comprising ex-vivo exposing the cells to cytokines and ionophores. No short-term boosting, but rather long-term culturing of the cells prior to administration is taught. No boosting with neurotransmitters, or devices or kits for short-term T-cell boosting are disclosed.

The present application relates to a method for augmenting the ability of certain body cells for fighting disease. The invention further relates to mixing devices and also to a kit, for use in such method. The invention is particularly useful for augmenting the ability of T-lymphocytes (T-cells) to fight certain types of cancer and infectious diseases, and CD3-zeta expression, and is therefore described below with respect to such application, but it will be appreciated that the invention could be used for augmenting the ability of other types of blood cells, such as NK cells, to fight disease.

### OBJECTS AND BRIEF SUMMARY OF THE PRESENT INVENTION

An object of the present invention is to provide a method particularly useful in the above-described technique for boosting and augmenting the ability of T cells, NK cells or other body cells for fighting disease, which enables the mixing the T-cells or other cells with T-cell boosting molecules, and especially their exposure to each other during a desired exposure period, to be conveniently made and maintained under sterile and tissue culture conditions. The T-cells, NK cells or other cells to be boosted are either naïve fresh un manipulated T-cells, NK cells or other cells, or T-cells that were pre pre-processed for days-months in-vitro for augmenting their anti-tumor-specificity, anti-virus-specificity, anti-bacteria specificity or other, and for their expansion.

The method described here also allows improving the effectiveness of various vaccines (anti-cancer, anti-viral, anti-bacterial, anti-parasite and others), by boosting and strengthening ex vivo the function of the respective anti-cancer/viral/bacterial/parasite T-cells, in a short and safe way, and at ant frequency needed.

The method described here allows also for marked upregulation of the CD3zeta expression in T-cells, which is crucial for T-cell activity against cancer of any infectious organism. T-cell of patients with various types of cancer have markedly decreased CD3zeta expression in T-cells, and CD3zeta expression levels correlated with prognosis and survival, and are used to monitor clinical trials in cancer.

The method described here allows also for marked upregulation of several other key receptors (e.g. CXCR, CD147 and 67-kDa LR) in T-cells, allowing significant augmentation in T-cell homing and invasion into diseased tissues.

Another object of the present invention is to provide T-cell boosting devices/kits, called herein ‘Boost-iT’, particularly useful for boosting T-cells, NK cells and other cells in such a method; and a further object is to provide a kit which includes one or more T-cell (or other cell) boosting devices for use in such boosting method.

Thus, by providing devices having compartments for containing desired type and amounts of T-cells, NK cells or other cells preselected portions of the substance and means for contacting and mixing T cells or other cells with substances under sterile conditions, the methods and devices of the present invention constitute a significant improvement over the prior art methods.

According to one aspect of the present invention, there is provided a method of augmenting T-cells, NK cells and certain other body cells of a patient for fighting disease in the patient. The method comprises exposing, for a predetermined exposure period, a first substance of cells of the patient having the ability to fight disease, to a second substance having a cell-boosting capability to augment the ability of the cells to fight the disease; and following the predetermined exposure period, re-introducing the first substance of boosted cells either back into the blood of the subject; or for further testing in vitro, or for freezing, or for any other use.

The method is further characterized in that the first substance is exposed to the second substance for the predetermined exposure period by mixing the two substances in a mixing device providing hermetically-sealed conditions effective to maintain sterility in both substances during the mixing/boosting, and appropriate tissue culture conditions (e.g. the desired CO2 concentration and humidity) thereof and for the predetermined exposure boosting period.

As indicated earlier, the method is particularly useful when the separated cells of the first substance are cells derived from the blood, lymph organ, tumor or any other organ, preferably T-cells, and the second substance is one having a capability to augment the ability of the cells to fight the disease, to elevate T-cell invasion of diseased tissue (e.g. an organ/tissue containing tumor) and elevate the level of expression of CD3 zeta in T-cells, for any purpose, including augmenting the T-cell elimination of cancer and infectious organisms. The T-cells can be separated or in whole cell preparations. In one preferred embodiment, the cells are isolated from the patient. In another embodiment, the cells are freshly isolated cells or stored cells.

According to one preferred embodiment of the invention described below, the cells are freshly isolated from an individual vaccinated against a disease, and the T-cells boosting is performed to augment the effectiveness of the vaccination.

According to yet another preferred embodiment of the invention described below, the method further comprises the step of assessing at least one cellular parameter in the cells, wherein the change in the cellular parameter is indicative of augmented ability of the cells to fight disease. The cellular parameters can be any of T-cell invasion into specific organs/tissues, extravasation, depolarization, integrin activation, de novo synthesis of specific genes, secretion of specific cytokines (especially TNFα and IFNγ), upregulation of CDzeta levels, upregulation of CXCR4, CD147, 67-kDa laminin receptor, T-cell receptors, upregulation of T-cell adhesion to fibronectin and laminin, chemotactic migration, and cancer cell killing.

According to one preferred embodiment of the invention described below, the first substance of T-cells or any other cells is mixed with the second substance by:

including the second substance in a dispensing port of a mixing device, the drug dispensing port having a first compartment, the first compartment having first and second needle-pierceable walls on its opposite sides; and including the first substance of cells in fluid communication with a second compartment of the drug dispensing port located on one side of the first compartment to face its first needle-pierceable wall, wherein the second compartment is movable from a normal position to an actuated position with respect to the first compartment and carrying two hollow needles facing the first needle-pierceable wall but spaced therefrom in the normal position of the second compartment, the first of the needles having a length to pierce both walls of the first compartment in the actuated position of the second compartment, and the second of the needles having a length to pierce the first, but not the second wall of the first compartment in the actuated position of the second compartment; and providing a mixing compartment in the mixing device located on the opposite side of the first compartment to face the second needle-pierceable wall thereof, and moving the second compartment to its actuated position to cause said first hollow needle to pierce both of said needle-pierceable walls of said first compartment and said second hollow needle to pierce said first wall of said first compartment; wherein the first hollow needle has a pointed open end and a side opening spaced from the pointed open end such that, in the actuated position of the second compartment, the side opening is located within the first compartment, and the pointed open end is located in the third compartment; wherein the second hollow needle has a pointed open end spaced so as to be located within the first compartment in the actuated position of the second compartment, and an upper opening spaced so as to be in fluid communication with the first substance of cells located within the second compartment in the actuated position of the second compartment.

According to further features in the described preferred embodiment, the mixing compartment is a cell chamber for mixing the first substance of cells and the second substance for the predetermined exposure period.

According to yet further features in the described preferred embodiment, the mixing device comprises receptacle having a plurality of mixing compartments.

According to another preferred embodiment of the invention described below, the first substance of cells is mixed with the second substance of cells by:

including one of said substances in a first compartment of a mixing device, the first compartment having first and second needle-pierceable walls on its opposite sides, including the other substances in a second compartment of the mixing device located on one side of the first compartment to face the first needle-pierceable wall thereof, wherein the second compartment is movable from a normal position to an actuated position with respect to the first compartment and carries two hollow needles facing the first needle-pierceable wall but spaced therefrom in the normal position of the second compartment, the needles having a length to pierce both walls in the actuated position of the second compartment, and providing a third compartment in the mixing device located on the opposite side of the first compartment to face the second needle-pierceable wall thereof; and moving the second compartment to its actuated position to cause both hollow needles to pierce both needle-pierceable walls of the first compartment, where one of the hollow needles has a pointed closed end, a first side opening spaced from the pointed closed end so as to be located within the first compartment in the actuated position of the second compartment, and a second side opening spaced from the pointed closed end so as to be located within the second compartment in the actuated position of the second compartment, while the other hollow needle has a pointed open end and a side opening spaced from the pointed open end such that, in the actuated position of the second compartment, the side opening is located within the second compartment, and the pointed open end is located in the third compartment.

In the described preferred embodiment, the second substance having the cell-boosting capability is included in the first compartment, and the first substance of separated cells is included in the second compartment.

According to yet further features in the described preferred embodiment, the cell boosting capacity comprises CD3 zeta elevating capacity.

Other embodiments are described wherein the first substance is mixed with the second substance by the substance having the cell-boosting capability in a compartment having an injection port and an outlet port; and injecting the cells into the compartment through the injection port.

In one described embodiment, the substance having the cell-boosting capability is included in each of a plurality of compartments interconnected in series by one-way valves with each compartment including an injection port, such that the quantity of the cell-boosting substance to be exposed to the cells may be selected as desired by injecting the cells in the injection port of a selected compartment containing the cell-boosting substance.

In another described embodiment, the two substances are mixed by: providing the cell-boosting substance in the form of a capsule; introducing the capsule in a compartment; rupturing the capsule by the application of pressure thereto; and injecting the cells into the compartment for exposure to the cell-boosting substance.

In yet another described embodiment, the second substance is provided in a plurality of capsules each introduced into a plurality of compartments, a capsule in one or more selected compartments is ruptured, and the first substance of cells is injected into each compartment where a capsule is ruptured to thereby enable augmenting the disease fighting ability of the desired selected quantity of the cells of the first substance. The compartments can be included in a plastic bag having internal partitions dividing the interior of the bag into a plurality of compartments, each having a capsule containing the second substance. The plurality of compartments can be arranged in series, with each compartment except for the last, having an outlet port communicating with the interior of the next compartment in a series via a one-way valve. The plurality of compartments, where arranged in series, can have, or be marked to indicate, an inlet opening at one end of the compartment, and an outlet opening at the opposite end of the compartment.

According to yet another aspect of the invention described below, there is provided a mixing device for mixing the first substance of cells and second substance in a hermetically sealed manner, the mixing device comprising a first compartment for one of the substances, the first compartment having first and second needle-pierceable walls on its opposite sides; a second compartment for the other of the substances located on one side of the first compartment facing said first needle-pierceable wall thereof and movable from a normal position to an actuated position with respect to the first compartment; two hollow needles carried by the second compartment facing the first needle-pierceable wall thereof and of a length to pierce both walls in the actuated position of the second compartment; and a third compartment located on the opposite side of the first compartment facing the second needle-pierceable wall thereof; wherein moving the second compartment to its actuated position causes both hollow needles to pierce both of the needle-pierceable walls of the first compartment, where one of the hollow needles has a pointed closed end, a first side opening spaced from the pointed closed end so as to be located within the first compartment in the actuated position of the second compartment, and a second side opening spaced from the pointed closed end so as to be located within the second compartment in the actuated position of the second compartment.

The third compartment can further include an outlet normally closed by a valve but openable after a predetermined exposure period to outlet the mixed substances. In one preferred embodiment, the predetermined period is one hour, preferably 45 minutes, more preferably 30 minutes.

According to yet another aspect of the invention described below, there is provided a mixing device for mixing the first substance of cells and the second substance in a hermetically sealed manner, the mixing device comprising a receptacle having a plurality of mixing compartments arranged in a series, each for receiving a quantity of one of said substances, each of said compartments having a drug dispensing port, a fluid inlet port and an outlet port, the outlet port of each compartment being connected to a one way valve permitting flow of the mixed substances into an outlet pipe, where the drug dispensing port comprises a first compartment for the second substance, the first compartment having first and second needle-pierceable walls on its opposite sides, and a second compartment in fluid communication with the first substance of cells located above the first compartment facing the first needle-pierceable wall thereof and movable from a normal position to an actuated position with respect to the first compartment; and a first and a second hollow needle carried by the second compartment facing the first needle-pierceable wall thereof, where the first hollow needle is of a length to pierce both walls in the actuated position of the second compartment, and the second hollow needle is of a length to pierce the first but not the second walls in the actuated position of the second compartment, and wherein the mixing compartment is located on the opposite side of the first compartment facing the second needle-pierceable wall thereof; and moving the second compartment to its actuated position causes both hollow needles to pierce the first needle-pierceable wall of the first compartment and the first hollow needle to pierce both needle pierceable walls of the first compartment, and wherein the first hollow needle has a pointed open end, a first side opening spaced from the pointed open end so as to be located within the first compartment in the actuated position of the second compartment, the pointed open end spaced to be located in the mixing compartment in the actuated position of the second compartment, and the second hollow needle has a pointed open end spaced so as to be located within the first compartment in the actuated position of the second compartment, and an upper opening spaced so as to be in fluid communication with the first substance of cells located within the second compartment in the actuated position of the second compartment.

According to further features in the described preferred embodiment, the mixing compartments are separated by a non-removable separator. In yet further features of the described preferred embodiment, the compartments are separated by a removable separator.

According to other aspects of the present invention, there are provided various constructions of mixing devices for use in the above-described method, and also a kit including one or more such mixing devices for use in the above-described method.

Further features and advantages of the invention will be apparent from the description below.

### BRIEF DESCRIPTION OF THE DRAWINGS

The invention is herein described, by way of example only, with reference to the accompanying drawings, wherein:

FIG. 1 is a three dimensional view of one form of the mixing device, the “MiNeueT Complete Boost-iT” for performing the method of augmenting certain body cells for fighting disease in accordance with the present invention;

FIG. 2 is a three-dimensional view detailing five of the components of the mixing device shown in FIG. 1;

FIG. 3 is a three-dimensional view detailing the drug dispenser port (102) of the mixing device shown in FIG. 1.

FIG. 4 schematically illustrates one embodiment of the mixing device of FIG. 1, having a non-removable separator (112) between the chambers (106);

FIG. 5 schematically illustrates another embodiment of the mixing device of FIG. 1, without a separator between the chambers;

FIG. 6 is a three dimensional view illustrating another form of the mixing device, the “MiNeueT Adjustable Boost-iT”, constructed for use in accordance with the present invention;

FIG. 7 is a cross-sectional illustration of the mixing device of FIG. 6, showing the device in normal, unactuated condition;

FIG. 8 is a cross-sectional illustration of the mixing device of FIG. 6, showing the device in actuated condition;

FIG. 9 is a detail of the cross sectional illustration of the mixing device of FIG. 6, showing the flow path (arrows) of fluid through the drug tray (114) in the device.

FIG. 10 is a three dimensional view illustrating yet another form of the mixing device useful in the method of the present invention;

FIG. 11 is an exploded view of the missing device of FIG. 10;

FIGS. 12a and 12b illustrate the normal and actuated conditions of the mixing devices of FIGS. 10 and 11;

FIG. 13 schematically illustrates another mixing device constructed in accordance with the present invention;

FIG. 14 schematically illustrates yet another mixing device constructed in accordance with the present invention;

FIG. 15a is a graph (15a) illustrating the upregulating effect of short-term GnRH-II stimulation on expression of CD3 zeta in T-cells;

FIG. 15b is a table of individual responses of subjects to GnRH-II stimulation;

FIG. 16 is a graph illustrating the upregulating effect of short-term (30 minutes) dopamine stimulation on expression of CD3 zeta in T-cells;

FIG. 17 is a graph illustrating the upregulating effect of long-term dopamine stimulation on expression of CD3zeta in T-cells;

FIGS. 18a-18h are graphs illustrating the triggering of robust TNFα secretion by Dendrotoxin-K (DTX-K) blockage of Kv1.1-subunit containing channels in normal human T-cells. FIG. 18a shows the effect of 24 hours exposure to 12 different ion channel blockers on TNFα secretion in T-cells. Highest concentrations used are indicated on the axis. Bars represent the average fold increase±SD of at least 2 individual determinations.

FIG. 18b demonstrates the dose-dependent character of dendrotoxin-K (DTX-K) induction of TNFα secretion (expressed as fold increase) in fresh human T-cells, following 24 hours exposure.

FIGS. 18c-18h shows the mean concentrations of TNFα (in pg/ml) secreted to the culture medium by normal peripheral T-cells of six subjects, in response to 24 hours exposure to 100 nM DTX-K. TNFα secretion is also expressed as fold increase for T-cells from subjects 1-3.

FIG. 19 is a block diagram illustrating the method of augmenting certain cells of the body for fighting disease and upregulating CD3zeta expression levels in accordance with the present invention.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more detail than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

### The Method

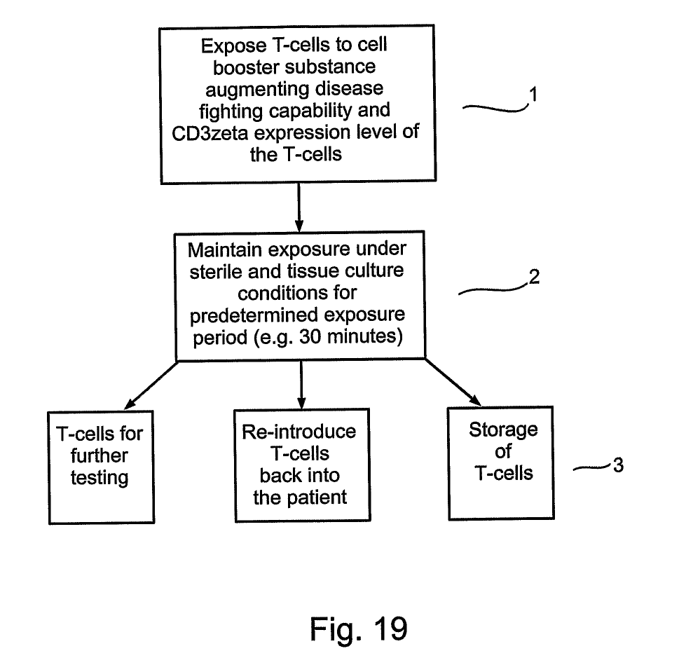

The method involved in the present invention is broadly illustrated by the flow-chart of FIG. 19.

Thus, as shown in FIG. 19, the method involves exposing T-cells separated from a blood portion, from primary or secondary lymph nodes, bone marrow and other hematopoietic tissue, or from a tumor-bearing tissue (i.e. biopsy) to a booster substance capable of augmenting the disease-fighting capability of the separate cells (block 1), or capable of elevating CD3 zeta expression in T-cells; maintaining this exposure for a predetermined exposure period, e.g. preferably short-term (up to 30 minutes) (block 2); and either reintroducing the so-treated T-cells into the patient, taking the so-treated T-cells for further testing (for changes in, for example, CD3zeta or TNFalpha expression levels), or storing the T-cells(blocks 3).

As indicated earlier, while the flow-chart of FIG. 19 and the further description below refer particularly to T-cells, it will be appreciated that the method and devices described can be applied for augmenting the disease-fighting ability of other blood cell populations, such as bone marrow. The method and devices described below are also not to be restricted to any particular short time periods since they may be used for boosting time periods if desired.

It will be appreciated that the T-cells or other cells suitable for use in the methods and devices/kits of the present invention can be provided in a number of ways.

Separation of T-cells for further boosting within the device, from either the blood, lymph nodes, tumor-bearing tissue or any other tissue any organ may be performed by well known methods, for example, density gradient separation (e.g. Ficoll™), cell sorting (e.g. FACS) and affinity- or immune-separation (e.g. CD4+, CD34+ selection), or any combination thereof. One preferred method for providing the T-cells or any other blood cells is plasmapheresis or leukopheresis in which specific blood components are removed ex-vivo from blood while it is being recirculated from the patient's circulation. In such systems, all the other blood cells besides those that were removed, are returned to the individual. Such blood components, such as T-cells, can then be then boosted by the devices and methods of the present invention.

Typically, blood cells are provided in flexible blood bags, such as, but not limited to the polyolefin Baxter Blood-Pack (Baxter Inc, Deerfield, Ill.) bags. Removal of blood cells from such blood bags for exposure to a substance can be accomplished only with significant danger of contamination, cell damage and waste. Similarly, introduction of a substance for mixing with the blood cells entails the risk of contamination and unintended puncture of the blood bag.

Substances can be added to the contents of the bag via an IV-type line, however this typically involves attachment of the IV line and a fluid reservoir containing the substance, or attachment of a connector, such as the cartridge described by Zbed et al in U.S. Pat. No. 4,804,366 and introduction of the substance into the connector in fluid connection with another fluid reservoir. Such connectors, however, do not provide for mixing of the beneficial substance with cells for a predetermined period of time, and are mostly designed to deliver a predetermined amount of substance to the IV line, in a dropwise manner, over a period of time. In one of the devices described herein; the ‘Complete Boosting Boost-iT device’ a complete boosting bag and kit is provided, having all the necessary elements to maintain the cells and boost them with the boosting drug in sterile conditions and in appropriate tissue culture conditions. In the other T-cell boosting devices described herein, ‘Adjustable Boost-iT device’ is described, containing the boosting drug and appropriate means to deliver the drug at the correct/desired/fixed concentrations into standard infusion bags, in sterile, consistent and controlled manner, without introducing any additional liquid or solution into the bag. Both types of devices allow accurate and reproducible mixing of the cells with the boosting substances.

Further, preparation of a substance for mixing with the T-cells or other cells, such as the neurotransmitters of the present invention, entails complex measuring, dilution and delivery steps which afford additional opportunity for contamination and errors. Yet further, in the interest of patient safety and accuracy of treatment, boosting of T- or other cells should always be carried out in a reproducible manner, using accurately measured concentrations, and preferably with molecules or compounds from the same batch, for uniformity.

Thus, by providing devices having compartments for containing desired type and amounts of blood cells, preselected portions of the substance and means for contacting and mixing T cells or other cells with substances under sterile conditions, the methods and devices of the present invention constitute a significant improvement over the prior art methods.

It will be appreciated that, in some embodiments, mixing devices for the method of the present invention can comprise compartments or chambers for both the substances, e.g. a blood cell chamber or compartment and a second compartment or capsule for the substance to be mixed with the blood cells. Such devices can be used for initial collection of the blood cells, eliminating the need for handling and transfer of the blood cells prior to the mixing steps. Such a chambered device can be designed for use with a plasmapheresis device, for automated collection and treatment of the collected cells, for example, while the patient and/or donor is in fluid communication with the plasmapheresis device. In one preferred embodiment, the blood cell chamber is about 500 ml in volume, subdivided to five (5) 100 ml subchambers, for mixing of up to 100×10⁶ blood cells with a substance. Suitable materials for construction of the device can be flexible, sterilizable, and permeable to CO₂ and other gases, such as the polyolefin blood bags available from Baxter, Inc. (Deerfield, Ill.).

In certain cases, it would be advantageous to deliver the substance for mixing with cells to cells already stored in a cell bag or similar cell collecting device. Thus, in another embodiment, there is provided a mixing device lacking a cell reservoir, comprising the substance for mixing in a sealed cartridge, means for fluid communication with, for example, an external cell reservoir or bag, and means for transferring the substance for mixing with the cells to the external cell reservoir or bag, for mixing. Thus, for example, the mixing device of the present invention can be used to deliver a T-cell boosting substance to cells collected in a prior-art blood cell collection bag.

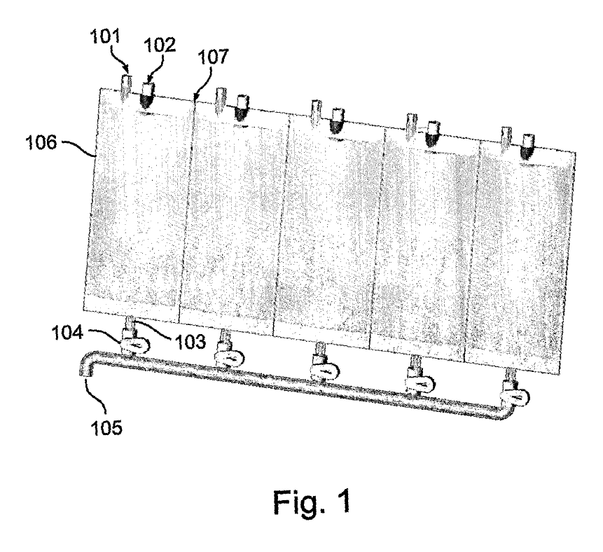

FIGS. 1-14 illustrate various types of mixing devices which may be conveniently used in the method of FIG. 19 for facilitating mixing the substance of separated cells with the substance having the cell-boosting capability under sterile conditions, and particularly for maintaining such sterile and tissue-culture conditions in both substances not only during the mixing thereof, but also during any required exposure time.

The methods and devices of the present invention can be used to boost T-cells for the treatment and prevention of

The Mixing Device of FIGS. 1-5

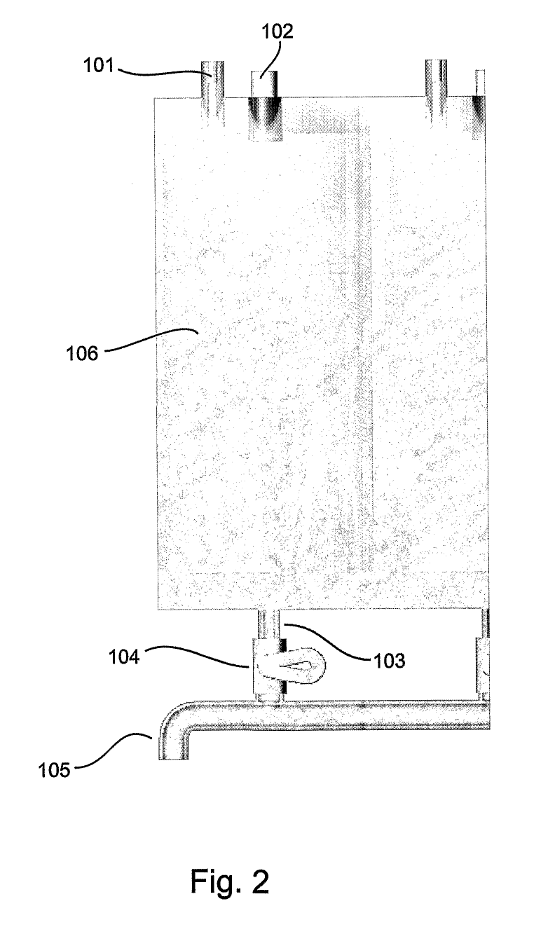

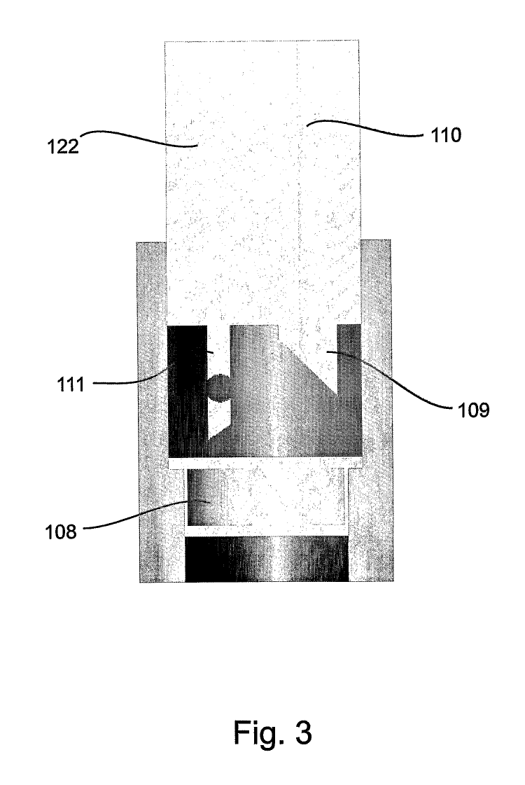

Device Description: The device of FIG. 1 contains 5 separate chambers 106, each with a capacity of 100 ml. At the top of each chamber are an inlet port 101 and a drug dispensing port 102. At the bottom is an outlet 103 which has a tap 104 on it to control the flow. Each of the 5 chambers then flow into a master outlet pipe 105. The chambers are separated from one another by a separator 107. FIG. 2 details the parts of the device described above: Inlet Port 101 is a simple inject site for the input of fluid; 100 ml chamber 106 is a water tight chamber with a capacity of 100 ml; Tap 104 is a standard on/off tap to allow or stop flow through it; Outlet Pipe 105 will be connected to the patient; Drug Dispensing Port 102 is for dispensing of the drug, and is described in detail in FIG. 3 below.

FIG. 3 details the Drug Dispensing Port: This works on the same principle for the MiNeueT adjustable device, there is a plunger which activates a first needle 109, which opens a drug tray 108 containing a medicinal substance. There is a second needle 111 which will then allow the infusing fluid to pass through the drug tray into the 100 ml chamber. The only difference is that inlet 110 is on top of the device to reduce size.

In FIG. 3 plunger 122 is in the start phase. The inlet 110 runs thought the plunger allowing access from the top for the fluid. When the plunger is depressed, the first needle 109 will allow fluid to pass into the drug tray 108 and second needle 111 will allow fluid to pass from the drug tray 108 into chamber 106 below.

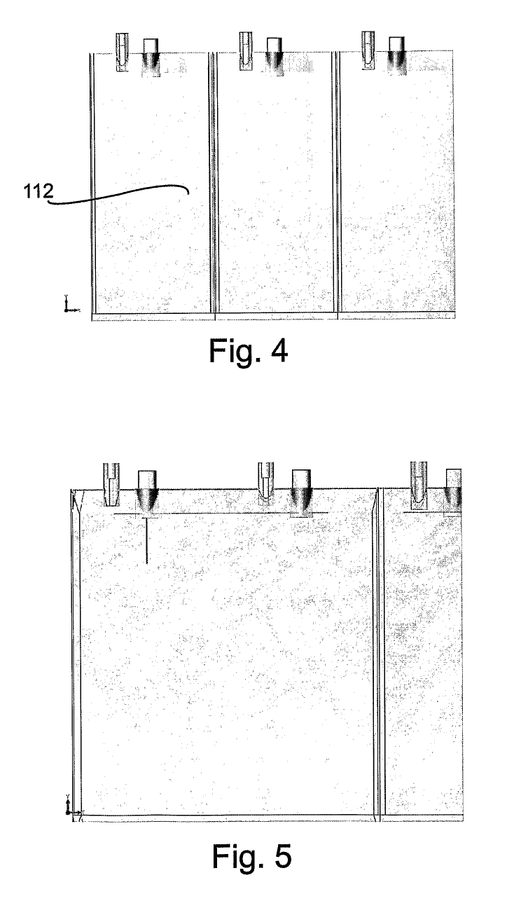

FIGS. 4 and 5 illustrate other embodiments of the mixing device of FIG. 1. FIG. 4 illustrates the mixing device having five independent 100 ml chambers separated by a separator 112. FIG. 5 illustrates a similar mixing device second designed to allow the separator to be breached.

FIG. 5 shows the mixing device of FIG. 1, once one of the separator walls 112 between the chambers has being removed. This will allow, for example the two 100 ml chambers to become a 200 ml chamber. Removal of the separators 112 between the chambers can be applied to all the internal walls, allowing the formation of a chamber of 100, 200, 300, 400 or 500 ml volume.

The following were incorporated into the device to reduce the failure modes and improve function and safety:

1. One device will incorporate a range of T-Cell volumes.

2. This device will wash that chamber holding the boosting powder will the T-Cells; this will insure a better mixture between the fluid and powder.

3. The device is designed to be simple to use.

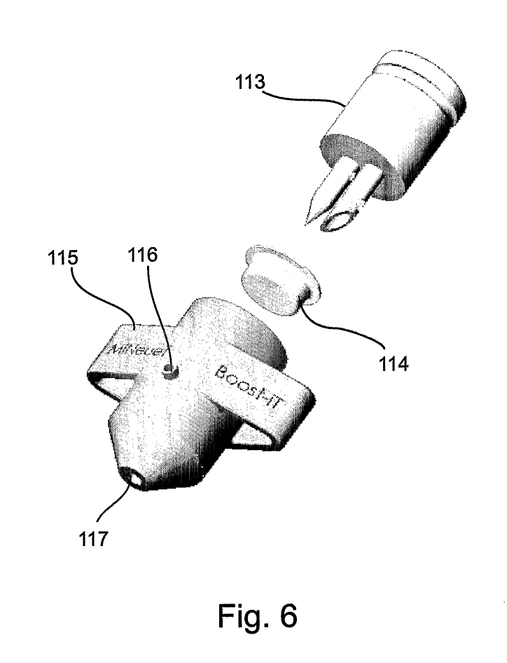

The Mixing Device of FIGS. 6-9:

Device Description: The mixing device of FIG. 6 is made up of three parts, a Case 115, Drug Tray 114 and Plunger 113. Case 115 is the housing which holds all of the parts together, on its top surface there will be an inlet port 116 for fluid to enter the device. At the distal end there will be an outlet port 117 to fit a standard blood bag. Drug Tray 114 is a fully sealed tray with lid which will contain the medicinal powder for the infusion into the blood plasma. Plunger 113 is a hand pushed plunger which will pierce through the drug tray before the fluid is entered into the device. It will also direct the fluid around the drug tray 114.

FIG. 7 illustrates the normal, unactuated state of the device of FIG. 6. Outlet 117 of the device will be connected to a standard blood bag, or directly to the patient. Inlet port 116 will have a syringe connected to it. In this step the fluid cannot pass through the device, insuring against failure modes.

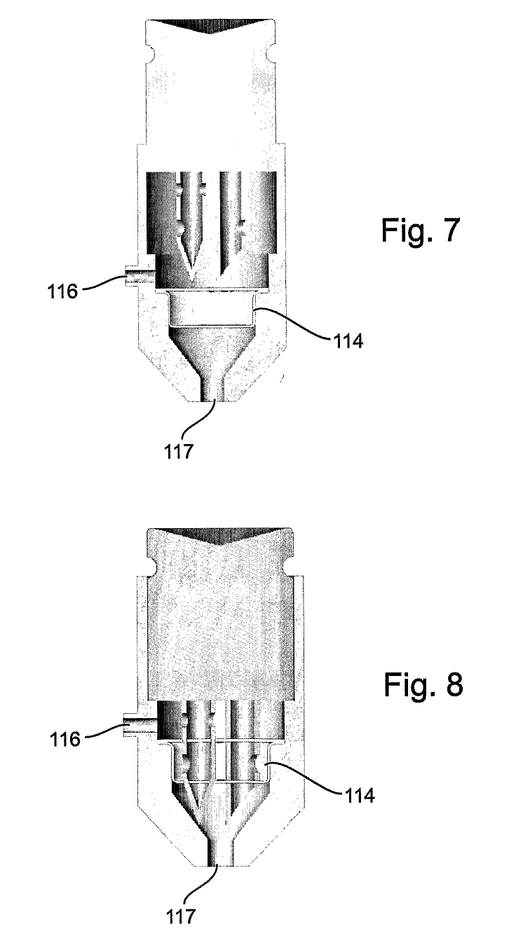

FIG. 8 illustrates the actuated state of the device when Plunger 113 is depressed, opening the drug tray 114 by ways of piercing. This will allow the fluid to not only pass into the drug tray 114, but also through the device to outlet 117 at the bottom.

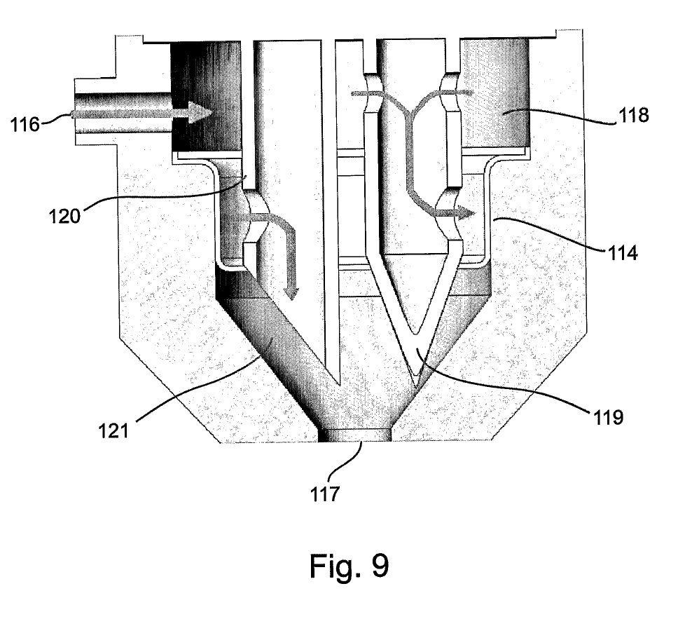

FIG. 9 illustrates the flow path in three steps. On the top left the fluid flows from inlet 116 into the upper chamber 118 of the device. On the right-hand side there is a first needle 119 which allows flow of fluid from the upper chamber 118 into the drug tray 114 where the T-cells and medicinal powder are mixed. Here the fluid will have to travel within this tray (indicated by the third arrow on the left side) until reaching a second needle 120. This second needle 120 allows the fluid to pass from the drug tray to the outlet chamber 121. From here the fluid will pass into the blood bag via the outlet port 117.

The following were incorporated into the device of FIGS. 6-9 to reduce the event of failure modes and improve function and safety.

1. The Medicinal Substance is in a fully sealed pouch (drug tray 114), insuring no leaks of the powder will occur during transport and other factors the device will be exposed to.

2. The device is made using the minimal number of parts to insure cost effective manufacture.

3. Rather than opening a door to the chamber holding the drug, this device will wash that chamber will the blood plasma; this will insure a better mixture between the fluid and powder.

4. The device is designed to be simple to use,
Step One: Connect the device to bag and syringe.
Step Two: Push the Plunger on the device
Step Three: Feed liquid through the device.

5. There will be a range of devices, having a variety of combinations of boosting substance and amounts, in order to provide greatest flexibility for the practitioner; the idea is the correct device would be selected for the specific amount of T-cells recovered, and for the specific treatment desired. Each device would be a clearly labeled (for example, a different colour) and would have the amount of T-cells stated on the case. The one in FIG. 6 shows the device to be used if 100 ml of T-Cells are recovered.

Further, in another embodiment, a filter could be placed on the outlet 117 of the device. The process of piercing the (for example, plastic) drug tray may cause a small piece of the material of the tray to break off. This will need to the trapped, and prevented from contacting the T-cell mixture. Also if a selective filter can be specified which will block un-infused booster molecules but allow infused T-cells through this could reduce failure modes.

The outlet of the device will need a connection method to a collecting bag, such as a blood bag. This device can also connect to a standard blood bag, or directly to the patient. In one embodiment, a barb is used if the connection is to a blood bag, or a luer connector is used if the connection is directly to a patient.

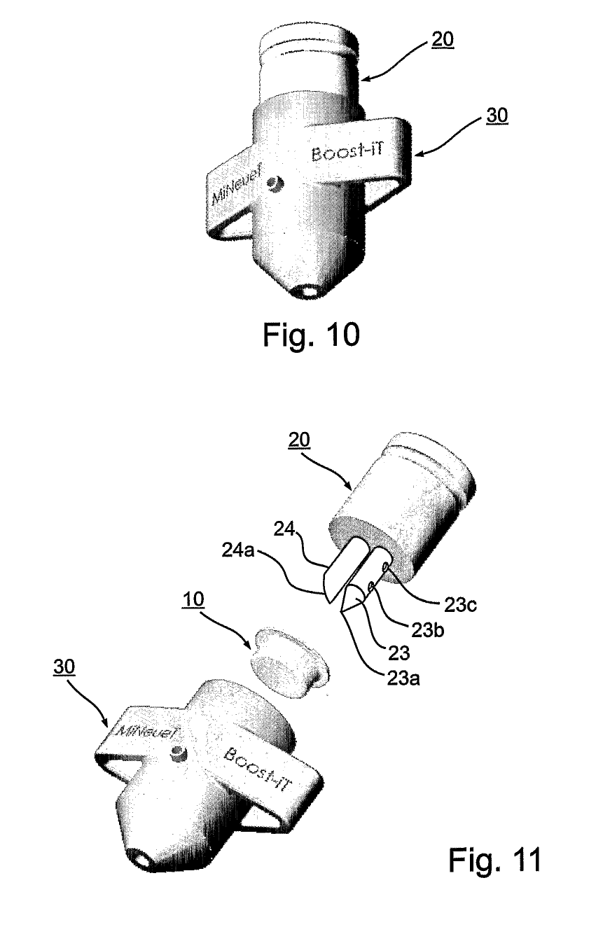

The Mixing Device of FIGS. 10-12a and 12b:

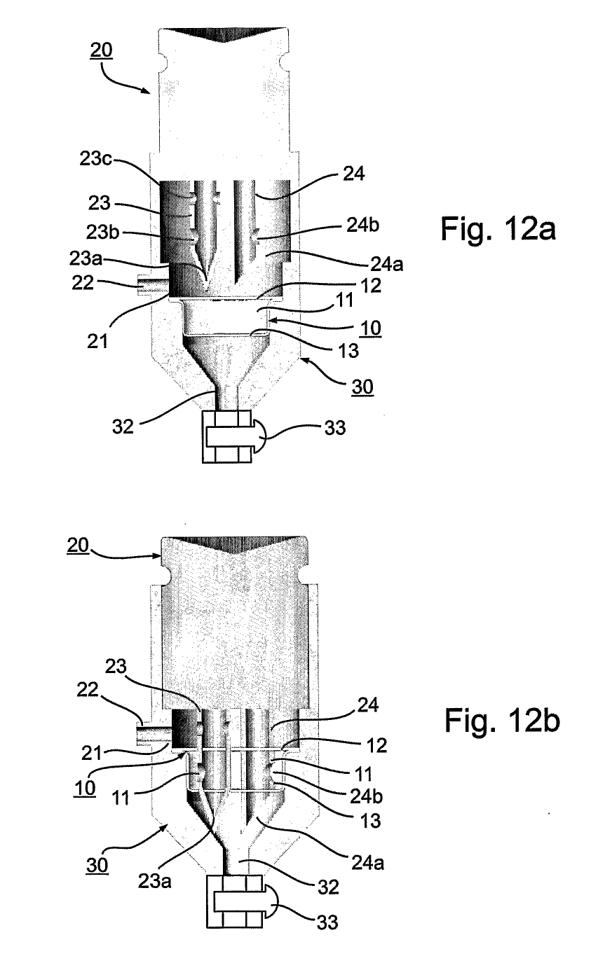

The mixing device illustrated in FIGS. 10-12b is constituted of three parts best seen in the exploded view of FIG. 11, and therein generally designated 10, 20 and 30, respectively. FIG. 10 illustrates the assembled condition of the mixing device, whereas FIGS. 12a and 12b illustrate the normal position and actuated position of its parts. As shown in FIG. 10, part 30 serves as a housing for receiving part 10, and then part 20.

FIG. 12a illustrates the normal condition of the mixing device, wherein it will be seen that part 10 defines a first chamber 11 to include one of the substances to be mixed, in this case the substance having the cell-boosting capability; part 20 defines a second chamber 21 to include or to receive the other substance to be mixed, in this case the separated cells; and part 30 defines a third compartment in which both substances are mixed together for a predetermined exposure time, preferably a short-term exposure, e.g. up to 30 minutes. As will be described more particularly below, the illustrated mixing device enables both mixing of the two substances, and maintaining their mixed condition for the desired exposure time, to be conveniently effected under sterile conditions. As shown in FIGS. 12a and 12b, part 30 defining the mixing chamber 31 is provided with an outlet port 32 closed by a stopcock 33 to allow outflow of the mixed substances after the predetermined time period, for further testing, storage, or re-introduction into the blood of the patient in accordance with the method illustrated in FIG. 19.

As shown particularly in FIGS. 11, 12a and 12b, part 10 defining chamber 11 for the substance having the cell-boosting capability is in the form of a sealed tray or cartridge pre-filled with the desired substance, which may be in powder or liquid form. It is closed on its opposite sides by needle-pierceable walls 12, 13, e.g., membranes of an elastomeric material. Such a cartridge thus defines a hermetically-sealed chamber 11 for the substance within it such that, after the cartridge and the substance are sterilized, the sterility of the substance is maintained until the hermetic seal is broken.

Part 20, defining chamber 21 for the second substance to be mixed, is in the form of a plunger moveable towards and away from cartridge 10. Plunger 20 further includes an inlet port 22 for introducing into chamber 21 one of the two substances to be mixed, in this case the separated cells.

The end of plunger 20 facing cartridge 10 carries two hollow needles 23, 24 having pointed ends facing the cartridge and of a length so as to penetrate both walls 12, 13 of the cartridge when the plunger is moved to its actuated condition, as shown in FIG. 12b. Pointed end 23a of hollow needle 23 is closed, whereas pointed end 24a of hollow needle 24 is open.

As shown particularly in FIGS. 12a and 12b, hollow needle 23 includes two side openings 23b, 23c; whereas hollow needle 24 includes a single side opening 24b. Side openings 23b and 23c in hollow needle 23 spaced from the closed pointed end 23a of the needle such that both side openings are within chamber 21 of the plunger 20 in its normal condition (FIG. 12a), but in the actuated condition of the plunger (FIG. 12b), side opening 23b remains within compartment 21 but side opening 23c moves into compartment 11 of cartridge 10, thereby effecting the mixing of the substance in compartment 21 with the substance in compartment 11. Side opening 24b in hollow needle 24, however, is spaced from its open pointed end 24a, such that in the normal condition of the plunger, side opening 24b is also within compartment 21 of the plunger (FIG. 12a), but in the actuated condition (FIG. 12b), side opening 24b is located in chamber 11 of cartridge 10, while the open pointed end 24a of needle 24 is located in chamber 31 of part 30.

It will thus be seen that in the normal condition of plunger 20, as shown in FIG. 12a, the substance within compartment 11 remains hermetically sealed within cartridge 10 by the two sealing membranes 12 and 13. However, when the plunger is actuated to its position illustrated in FIG. 12b, the pointed ends of both needles 23, 24 pierce both sealing membranes 12, 13 of the cartridge. Communication between compartments 21 and 11 is thus established by side openings 23b and 23c of needle 23, whereas communication between compartment 11 and compartment 31 of part 30 is established by side opening 24b of needle 24 and the open pointed end 24a of needle 24. Accordingly, actuating the plunger causes its two needles to pierce the two end walls 12, 13 of cartridge 11, while still effectively maintaining the hermetically-sealed conditions of the three compartments 11, 21 and 31, such as to substantially preserve sterility in the substances within those compartments for the required exposure time. After that, stopcock 33 may be removed to outlet the contents of compartment 31.

It will thus be seen that the substance having the cell-boosting capability may be included and hermetically sealed within cartridge 10 so as to be easily stored, transported and handled while maintaining sterility therein. When the substance is to be used in the method, as illustrated in FIG. 19, for augmenting the ability of body cells to fight disease, cartridge 10 would be introduced into the mixing device, between plunger 20 and part 30. Plunger 20 is moved from its normal condition (FIG. 12a) to its actuated condition (FIG. 12b). In the actuated condition of plunger 20, its needle 23 establishes communication between chamber 21 of the plunger and chamber 11 of the cartridge, and its needle 24 establishes communication between chamber 11 of the cartridge and the outlet chamber 31. Accordingly, cells separated from the patient for exposure to the cell-boosting substance within chamber 11 of cartridge 10, may be injected into chamber 21 via inlet 22. The injected cells pass, via side-openings 23b, 23c of hollow needle 23, into chamber 11 of the cartridge for mixing with the cell-boosting substance therein. The mixed substances may then pass, via side opening 24b and open end 24a of needle 24, into compartment 31, wherein the mixture is retained in the hermetically-sealed sterile conditions, for the required exposure time, e.g. up to 30 minutes. After the required exposure time has passed, stopcock 23 is removed to outlet the so-treated cells for further testing, storage or re-reintroduction back into the patient's body.

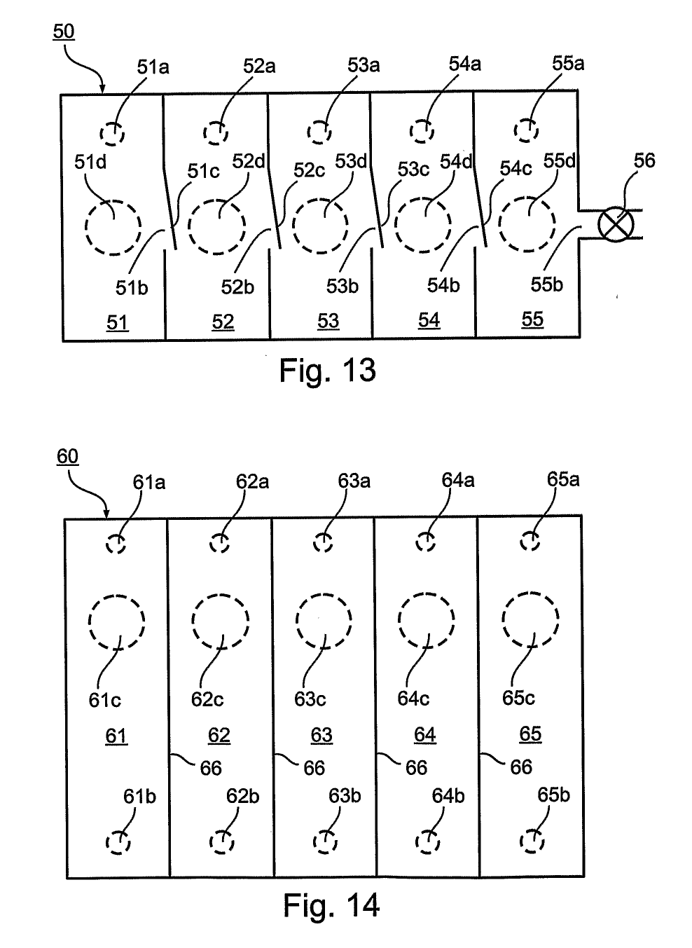

The Mixing Device of FIG. 13

FIG. 13 illustrates another mixing device which may be used in the method described above with respect to FIG. 19. In this case, the mixing device, generally designated 50, is in the form of a receptacle having a plurality of compartment 51-55 arranged in a series. Each compartment is capable of receiving a quantity of one of the substances to be mixed, in this case, the cell-boosting substance. Each compartment 51-55 includes an inlet port, as shown at 51a-55a, respectively, for inletting therein the other substance to be mixed, in this case the separated cells.

Each of the compartments 51-55 further includes an outlet port 51b-55b, respectively, connecting the interior of one compartment to the next compartment in the series, such that the interior of compartment 51 communicates with the interior of compartment 52, interior compartment 52 communicates with the interior compartment 53, etc. The outlet 55b of last compartment 55 in the series includes an outlet valve 56. Each of the outlets 51b-54b also includes a one-way valve, e.g., a pivotal or freely-mounted valve member 51c-54c outwardly of its outlet port, permitting fluid flow in only one direction, outwardly of its outlet port.

It will thus be seen that the mixing device illustrated in FIG. 13 can also hold the substance having the cell-boosting capability in a hermetically-sealed compartment for ease of transportation, storage and handling, and then used whenever needed for augmenting the disease-fighting ability of cells separated from the body in the manner described above with respect to FIG. 19. The mixing device illustrated in FIG. 13 further permits the cell-boosting substance to be supplied in different quantities such that the appropriate quantity can be selected for any particular application by merely introducing the separated cells through one or more of the inlets 51a-55a of the mixing device illustrated in FIG. 13. Such a device conveniently permits not only the quantity of separated cells to be selected, but also the quantity of the cell-boosting substance to be selected, for any particular application. For example, if it is desired to expose a large quantity of the cell-boosting substance to a relatively large quantity of the separated cells, the separated cells could be injected into several or all of the inlet ports 51a-55a. On the other hand, if it is desired to expose a relatively small quantity of separated cells to a relatively large quantity of the cell-boosting substance, the separated cells could be injected only into inlet opening 51a of the first compartment 51 of the series, and successively passed through all the compartments via the one-way valves 51c-54c of all the compartments into the last one 55, so as to be exposed to the quantity of substances in all the compartments. However, if only a relatively small quantity of separated cells is to be exposed to a relatively small quantity of the cell-boosting substance, the separated cells could be introduced only into the last compartment 55 of the series, via its inlet port 55a.

In any of the above cases, the separated cells and the cell-boosting substances may be retained in the last compartment 55 for the required exposure period, and then outletted via valve 55b.

It will also be appreciated that the compartments 51 could contain different quantities of the same substance, or different substances, such that separated cells introduced into the inlet of one compartment (e.g. 51a) would be successfully exposed not only to different quantities of the same type of substance, but also to different types of substances in the subsequent compartments 52-55 of the series.

A preferred construction of the mixing device illustrated in FIG. 13 would be to make receptacle 50 of a pliable plastic bag integrally formed with partitions to define the compartments 51-55 with an opening in each partition to define the outlet openings 51b-54b, and with each of the latter openings covered by a plastic flap to define the one-way valves 51c-54c. In such a construction, each compartment would include the cell-boosting substance in the form of a capsule 51d-55d. All the inlets 51a-55a, and also the outlet 55b of the last compartment, need not be formed at the time of production of the bag, but could be marked to facilitate their formation, by piercing, at the time of use of the bag.

The Mixing Device of FIG. 14

The mixing device of FIG. 14 is similar to that of FIG. 13, in that it may also include a plastic bag, generally designated 60, divided into a plurality of compartments 61-65 by partitions 66. With each compartment containing a quantity of one of the substances to be mixed, in this case the substance having the cell-boosting capability. Each compartment further includes an inlet port 61a-65a for injecting the second substance to be mixed, namely the cells separated from the patient's body for augmentation of their disease-fighting capability before re-introduction into the patient's body.

One difference in the construction of the mixing device illustrated in FIG. 14, over that illustrated in FIG. 13, is that all of the compartments 61-65 in the FIG. 14 construction are arranged in parallel with each compartment having a separate outlet 61b. As in the Fig. mixing device, the substance within each of the compartments 61-65 in FIG. 6 is preferably also enclosed within a capsule 61c-65c, respectively, which is manually rupturable in order to release the substance into its respective compartment. Thus, if a relatively small quantity of separated cells is to be exposed to a relatively small quantity of the cell-boosting substance, a capsule (e.g. 61c) for one compartment may be manually ruptured, and the relatively small quantity of separated cells may be injected via the inlet opening (61a) of the respective compartment (61), retained therein for the required exposure period, and then outletted via the respective outlet (61b). On the other hand, if a larger quantity of separated cells is to be exposed to a larger quantity of the cell-boosting substance, the same manipulations may be made with respect to two or more of the compartments of mixing device 60.

The mixing device illustrated in FIG. 14 could be similarly constructed of a plastic bag 60 formed with the partition 66 to define the compartments 61-65, with a capsule 61c-65c of the cell-boosting substance in each compartment, as in the above-described construction for the mixing device illustrated in FIG. 5. In the case of the mixing device of FIG. 14, however, the partitions would not be formed with the outlet openings and with the flaps defining one-way valves (51c-54d). Rather, the outlet opening 61b-65b of each compartment would be at the end of each compartment opposite to that of the inlet opening 61a-65a, as shown in FIG. 6. In such a construction, it would also be preferable not to form the inlet and outlet openings at the time of production of the bag, but rather merely to mark these openings on the bag, to facilitate their formation, by piercing, at the time of use of the bag.

It will thus be seen that the above-described mixing devices conveniently permit storage, transportation and handling of the cell-boosting substances; better assure sterility when exposing the separated cells to such substances to augment the ability of the separated cells to fight disease; permit long-term storage or freezing of the cell-boosting substances if desired; are disposable after one-time use; and may be produced in volume and at low cost.

It will be appreciated that monitoring of the response of T-cells or other cells, boosted by the devices and methods of the present invention, is advantageous, for example, in order to accurately assess the need for further boosting of the cells, to determine the proper amounts of cells required for re-introduction into the patient, to serve as a basis for further prognosis, and in order to allow evaluation of cells after storage and/or additional ex-vivo treatments. Thus, the T-cell responses to neurotransmitters, calcium channel blockers and other boosting substances detailed hereinabove can be used to provide an assessment of the response of the T-cells to boosting.

In one preferred embodiment, the method of the present invention comprises exposing, for a predetermined exposure period, the separated cells to a substance having a cell-boosting capability to augment the ability of the separated cells to fight the disease, and reintroducing the separated cells back into the subject, or transferring them to further in vitro testing or processing, or for freezing/crypreservation, characterized in that the cells are exposed to the boosting substance by mixing the cells and the substance in a mixing device providing hermetically-sealed conditions effective to maintain sterility in both the cells and the substance during the mixing thereof and for said predetermined exposure period. In a yet more preferred embodiment, a sample or samples of the cells are monitored following the mixing, to determine the level of a parameter, marker or function indicative of boosting of T-cell activity. Such parameters, markers or functions include, but are not limited to T-cell extravasation, depolarization, integrin activation, T-cell adhesion, de novo synthesis of specific genes, secretion of IL 10 and TNFalpha, upregulation of CDzeta levels, adhesion to fibronectin and laminin, chemotactic migration, and cancer cell killing. One can measure the desired marker, or markers, prior to storing or further treating the cells, for example, and compare the levels of markers in the treated cells to a standard or to untreated cells, in order to assess the effects of the treatment on the T-cells.

While reducing the present invention to practice, it was surprisingly uncovered that ex-vivo treatment of T-cells with neurotransmitters dopamine and GnRH-II results in increased levels of CD3zeta in the cells. Thus, according to a more preferred embodiment, the level of CD3zeta is determined by known assays of CD3zeta, in T-cells following treatment by the device and methods of the present invention.

As used herein, the term “boosting” of T-cells or other cells is defined as a strengthening of the desired, predetermined disease-resisting characteristics of the cells. The strengthening described herein calls for exposing, for a predetermined exposure period, and in sterile and appropriate cell culture conditions, the T-cells to a substance/molecule having a cell-boosting capability to augment the ability of the separated cells to fight the disease; and following the predetermined exposure period, reintroducing the treated boosted T-cells back into the blood of the subject, at any desired rate. Alternatively, at the end of the boosting process, the T-cells can be transferred and used for any other purpose, such as for further testing in vitro, for keeping the boosted cells for some time till they can be infused to the patients, or for freezing/cryo-preservation of the boosted T-cells for keeping ‘a personalized boosted T-cell bank’ for each patient. It will be appreciated that a patient can be any individual in need of “boosting” of T-cell activity, including all mammalian species. Thus, the methods and devices of the present invention are suitable for human clinical use, and for veterinary use, in addition to use in scientific experimentation.

Several methodologies currently used for eliminating cancer by tumor specific T-cells require storage (“parking”) of the patients autologous T-cells in vitro for days-months, needed for the respective genetic and other manipulations for making the T-cells tumor specific, and obtaining high numbers of such cells. Such procedures, long periods of in vitro parking and T-cell receptor (TCR) activation performed usually just before reinjecting the T-cells into the patients, hamper the ability of the TCR-activated tumor-specific cells T-cells to migrate in vivo, home in vivo, adhere in vivo etc, due to crucial loss of key receptors on the T-cell cell surface (e.g. CXCR4), and other undesired down regulations. Thus, the methods, devices and kits of the present invention, for boosting T-cells prior to administration, using such tumor-specific, or genetically modified T-cells, can be an effective adjunct to the ex-vivo manipulation of tumor-specific T-cells.

In the case of T-cells, the T-cell boosting is achieved within a device in which the patient's T-cells, either fresh or pre-processes in vitro for days-months before that, are exposed for relatively short time periods (in most cases up to 30 minutes) to T-cell boosting molecules, and then returned to the patient, with an augmented ability to fight the cancer or infecting organisms (virus, bacteria, parasite, prions etc) that invaded the patient's body and have deleterious effects on the patient's health.

While the present invention focuses on T-cells, it is clearly not restricted to these cells, and can also be applied to NK cells and other blood cell populations which can assist in fighting a disease. Interestingly, some key receptors in NK cells are also down regulated in cancer cells, (alike CD3 zeta in T-cells). Therefore, for example, the methods and T-cell boosting devices described herein may be used to boost, for example NK cell activity as well, with the expectation that the same procedures that elevate CD3zeta, would elevate key receptors in NK cells. Further, the procedures described below dealing with T-cells can be applied to any other blood cell population. The invention is also not restricted to short time exposures but allows longer boosting time periods if needed.

The T-cell boosting molecules are primarily (but not exclusively): (1) specific neurotransmitters, or their synthetic analogues that are neurotransmitter receptor agonists and antagonists, which have specific receptors expressed in T-cells; and which were found by M. Levite's to trigger by themselves, in physiological concentrations, numerous key T-cell features and functions (2) Particular highly selective ion channel blockers that regulate particular ion channels present on T-cells, and by doing activate potent T-cell functions, such as secretion of key cytokines. The neurotransmitters used in the device to boost the patient's T-cells upon short and direct exposure, include primarily (but not exclusively) Dopamine, Glutamate, GnRH-II, GnRH-I, Somatostatin, Calcitonin gene related peptide (CGRP), Neuropeptide Y (NPY) and Substance P. All these neurotransmitters and some of their highly selective receptor agonists were found to have the ability to augment or trigger T-cell functions on their own in the complete absence of any additional molecules. The ion channel modulators include primarily (but not exclusively) blockers of voltage-gated potassium channels, especially highly selective blockers of the Kv1.1 voltage-gated potassium channels, expressed in T-cells, such as dendrotoxin K, which by themselves were found by M. Levite to trigger robust, exclusive and prolonged production and secretion of tumor necrosis factor alpha (TNFα) (see Examples section hereinbelow). In addition to ion channel blockers, TNFα production and secretion, from T-cells and other cells, can also be triggered or augmented by specific anti-ion channel antibodies (such as the anti-kv1.1 channel antibodies described hereinbelow) and any other molecules effective in blocking signal transduction downstream of the ion channels.

A non-limiting list of suitable ion channel blockers includes 4258-v, MCD-Peptide, 4287-v, Stichodactyla Toxin, 4290-s, Margatoxin, 4313-s Tityustoxin Kα, 4330-s, Dendrotoxin I, 5′-Adenylyl-imidodiphosphate, Agitoxin-2,4-Aminopyridine, Apamin, BDS-I, Anemonia sulcata, BDS-II, Anemonia sulcata, rCharybdotoxin, Clotrimazole, Dendrotoxin Dendrotoxin I, Dendrotoxin K, βDendrotoxin, γ-Dendrotoxin, Dequalinium Chloride, Glyburide, Iberiotoxin, Kaliotoxin, Noxiustoxin, Paxilline, Penitrem A, SKF-525A, Tetraethylammonium Chloride, Tolazamide, Quinine, Clotrimazole, Tetrodotoxin, NPPB, R-(+)-Bay K8644 and CNQX. Channel blockers are available commercially, for example, Calbiochem, Inc. (San Diego, Calif.).

TNFα, a key pro-inflammatory cytokine with an extremely wide spectrum of activities, plays also a cardinal and beneficial role in health. It activates the innate immune response and induces essential inflammation, which protects the body from environmental attack. In addition, TNFα causes necrosis of tumors, augments rolling and adhesion of leukocytes, increases the permeability of blood vessels and the blood brain barrier (BBB) to various molecules, induces proliferation and regeneration of oligodendrocytes and promotes nerve remeyelination, recruits cells into specific tissues, among many other important functions.

The methods and devices of the present invention are suitable for use in treating all types of diseases where boosted T-cells, can be of advantage to fight the disease, comprising of cancer of various types, in all of which enhancement of anti-tumor immune surveillance is needed, viral and other infectious diseases, immunodeficiency diseases (genetic or acquired) or transient immunodeficiency conditions (e.g. after bone marrow transplantation, after infection, after major stress, after injury/accident, after stroke and others), treatment of neurological disease, neuropsychopathology, stress, neuronal healing and regeneration following disease or injury to the CNS. Immune deficient conditions and diseases that may be treated by the method and devices of the present invention include primary immunodeficiencies, such as the acquired immunodeficiency syndrome (AIDS), DiGeorge's (velocardiofacial) syndrome, adenosine deaminase (ADA) deficiency, reticular dysgenesis, Wiskott/Aldrich syndrome, ataxia-telangiectasia, severe combined immunodeficiency; and secondary immunodeficiencies, such as anergy from tuberculosis, drug-induced leukopenia, non-HIV viral illnesses leukopenia, radiation poisoning, toxin exposure, malnutrition, and the like. Similarly, neoplastic disease or conditions resulting from failure of immune surveillance, and bacterial, fungal and viral infections, especially of the CNS, brain-related injury, degeneration and psychopathology may be treated by the methods and devices of the present invention.

The methods and devices and/or kits of the present invention can also be used to treat conditions characterized by over-activity of T-cell function in the patient. Such conditions include, but are not limited to, T-cell mediated auto-immune diseases such as, for example, Rheumatoid Arthritis, Multiple Sclerosis Type-I diabetes, and autoimmune hyperthyroidism, and complications of transplants such as restenosis, graft versus host disease (GVHD) and host versus graft disease (HVGD), T-cell malignancies such as lymphoma and leukemia, diseases caused by cytolytic T-cells, such as certain forms of hepatitis, chronic inflammation, and allergic diseases. For treatment of such conditions of over-activity of T-cell function, a T-cell subpopulation of regulatory/suppressor CD4+ CD25+ T-cells, rather than the whole T-cell population, is boosted. Boosting the activity of such regulatory T-cell populations in turn reduces the activity of the overactive inflammatory T-cells.

It will be appreciated, that T-cell function and number decline with advancing age, resulting in insufficient immune competence and rendering the elderly susceptible to infection and attack by a wide variety of otherwise easily resisted organisms. Thus, the methods and devices described herein may be used for augmenting the immune competence and immune resistance of elderly individuals to infectious and parasitic disease, and to cancer of various types.

It is to be emphasized, however, that T-cell boosting within the device is not restricted to neurotransmitters or to ion channel modulators, but is achievable by any other molecules able to boost T-cell function, preferably those compounds found effective following short-term exposure.

The T-cell boosting devices and/or kits to augment the function of the patient's T-cells can be used at any frequency, as determined by the clinicians, for example once a day/week/month for prolonged periods of time. The use of the T-cell booster technique described herein is simple and safe: In one preferred embodiment, the patient's own T-cells, either freshly isolated from his blood or from his tumor-bearing tissue, or after days to months of pre-processing in vitro, for example, for augmenting their anti-tumor-specificity, anti-virus-specificity, anti-bacteria specificity or other and for their expansion, are exposed to natural/physiological neurotransmitters or their analogues and/or to ion channel modulators. Further, after exposure of the patient T-cells to these neurotransmitters, the device may wash the T-cells before their re-introduction back into the patient, or prior to any other use of these cells (e.g. crypreservation, further treatment or testing, etc before reintroduction into the patient). However, it will be appreciated that since the physiological neurotransmitters are used at relatively low physiological concentrations (±10 nM), and that their life time in vivo is usually and typically brief, the prior presence of the neurotransmitters with the T-cells is not expected to cause any problem in vivo, even if the cells are not washed prior to use. Thus, minimal side effects, if any, may be expected. Furthermore, since the neurotransmitters usually do not interfere with the TCR-specificity of the T-cells, and do not induce marked proliferation of the cells, no in vivo autoimmune activities/side effects are expected of the boosted T-cells.

Boosting of T-cell activity can be preceded by separation of T-cells by any of the conventional methods known in the art. The stage of T-cell boosting by neurotransmitters or ion channel modulators within the T-cell boosting device may be preceded, if needed, by a prior stage, outside the device, of separation, of the patient's whole T-cell population into T-cell subpopulations. In such an earlier stage, prior to entering the device, specific T-cell subpopulation can be positively selected, by various commonly used methodologies, and then transferred to the T-cell boosting device or negatively selected (i.e. CD4+ CD25+ regulatory T cells). In the case of negative selection, all other T-cell subpopulations (i.e. devoid of the negatively selected subpopulations) are transferred to the T-cell boosting device where they are exposed to neurotransmitters and/or their receptor analogues and/or particular ion channel modulators for short time periods. They are then returned to the patient to treat the disease [e.g. cancer and infectious diseases (virus, bacteria, parasite, prions etc)] via the boosted T-cell capability of doing so by the above-described exposure, or transferred to further uses (e.g. further tests and/or cryopreservation).

The method and device/kit used herein can serve also for improving the effectiveness of various vaccination protocols against cancer, viruses, bacteria and others. Vaccination protocols, especially anti-cancer vaccinations, suffer from disappointing results partly due the lack of sufficient T-cell homing to, and invasion into, solid tumors, due to the active counter attack of the T-cells by the cancer, due to the low CD3 zeta of the patient's anti-tumor T-cells which prevent productive recognition and elimination of the cancer, due to the fact that many anti-tumor T-cells are in poor viability state, and in fact are dying via, for example, apoptosis. In such cases, the boosting of the vaccinated individuals T-cells will be performed after vaccinated with a given antigen or antigens (by any methodology).

As indicated above, the exposure of the T-cells to the substance having the above-described cell-boosting capability may be for relatively short periods of time, in most cases up about 30 minutes. It will be appreciated that the ability to maintain conditions critical to tissue or cell culture, such as humidity and CO₂ balance, are desired, but that in any case the “boosting” must be done under conditions which maintain sterility in the two substances when mixed, and for the complete exposure period thereafter.

According to one embodiment of the present invention, there is also provided a kit for augmenting the disease-fighting capacity and tissue invasion capability and CD3zeta levels of cells, such as T-cells. Such a kit will comprise any of the abovementioned devices for performing the methods of the invention, packaged into a kit which can be used for treatment of cells. The kit for use in the method according to the invention preferably contains the various components needed for carrying out the method packaged in separate containers and/or vials and including instructions for carrying out the method. Thus, for example, some or all of the various ingredients needed for carrying out the determination, such as the devices, boosting substance(s), etc, can be packaged separately but provided for use in the same box. Instructions for carrying out the method can be included inside the box, as a separate insert, or as a label on the box and/or on the separate ingredient packages.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Neurotransmitters Upregulate CD3zeta Levels in T-Cells

As detailed hereinabove, markedly low CD3zeta levels in T-cells observed in various types of cancers and have been correlated with poor prognosis in some cancers. Low CD3zeta levels have also been observed in certain autoimmune and infectious diseases. Thus, the effect of exposure of T-cell cells to neurotransmitters known to trigger T-cell activity on CD3zeta levels was assessed, specifically in short-term treatment.

Materials and Experimental Method

Normal human T-cells, isolated from fresh peripheral blood lymphocytes, were treated with 10 nM of neurotransmitters: either GnRH-II or dopamine (30 min or 24 hr at 37° C.). Following this neurotransmitter treatment, the T-cells were washed with phosphate-buffered saline (PBS) and permeabilized with paraformaldehyde (PFA) for 20 min at room temperature (1×10⁶ cells resuspended in 50 μl PBS+100 μl 0.5% PFA). The T-cells were then washed again with PBS and subjected to single immunofluorescence staining, using a mouse anti-human TCR/CD3zeta chain subunit (also called CD247) mAb (Serotec) at 1:100 dilution/1×10⁶ cells, diluted in 10 μg/ml digitonin (Sigma) cold solution, for 30 min on ice. For isotype control, cells were stained with normal mouse serum (Jackson Immunoresearch Laboratories). The cells were then stained with a fluorescein isothiocyanate (FITC)-conjugated anti-mouse IgG (100 μl of 1:100 dilution; Jackson). Cells stained with the second Ab only served as additional negative controls. Fluorescence profiles were recorded in a FACSort.

Results

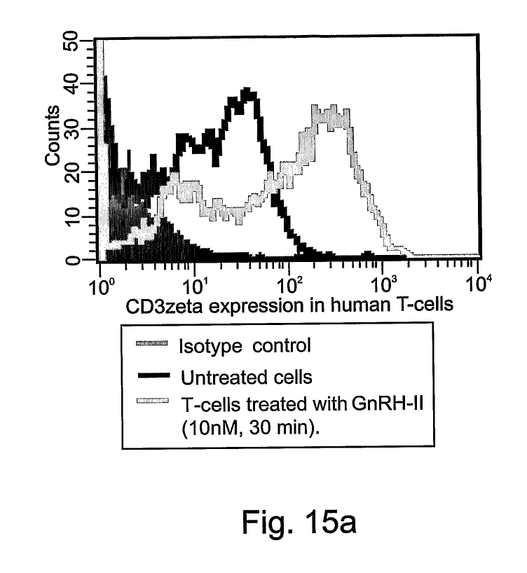

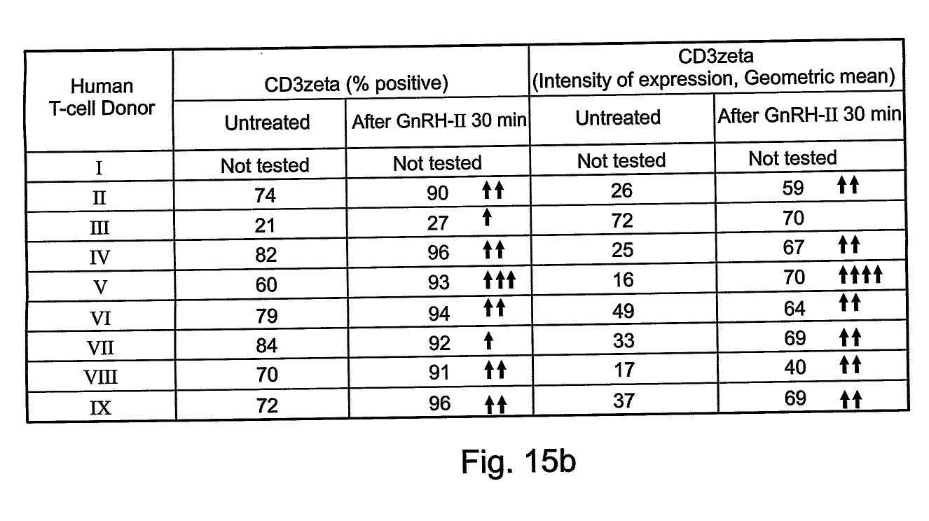

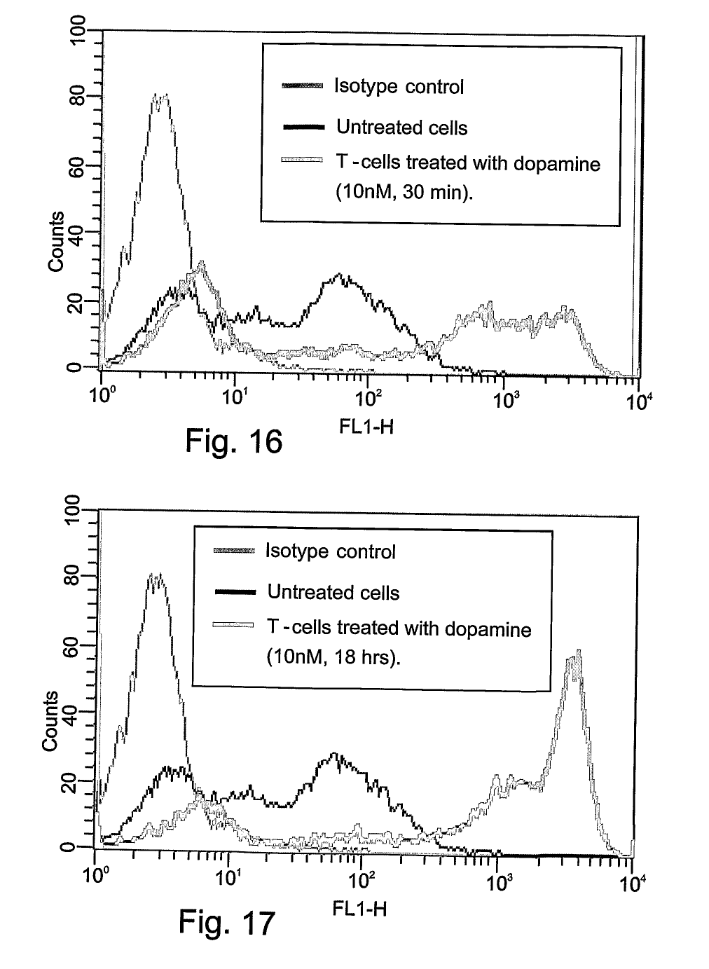

FIGS. 15-17 show the effect of short-term (FIGS. 15a, 15b and 16) and long-term (FIG. 17) exposure of human T-cells to physiological concentrations (10 nM) of neurotransmitters GnRH-II or dopamine (in the absence of any other molecules besides the respective neurotransmitter). 30 minutes exposure of T-cells to GnRH-II, as shown in FIG. 15a, clearly causes a very significant increase in both the number (percentage) of cells expressing CD3zeta, and in the intensity of CD3zeta expression (i.e. number of CD3zeta per T-cell) in the treated human T-cells (see individual subject's responses, FIG. 15b). 30 minutes exposure to dopamine also produced a clear increase in the number of normal peripheral human T-cells (i.e. T-cells separated from a ‘fresh’ human blood sample) expressing CD3zeta (FIG. 16), an effect which becomes significantly more pronounced with longer-term exposure (18 hr) to the neurotransmitter (FIG. 17).

Since reduced expression of CD3 zeta in T-cells has been suggested as an important prognostic marker in cancer (see Whiteside, et al, Canc Immunol Immunotherap 2004, 53:865-78), and the CD3 zeta levels in T-cells are “boosted” following exposure, even in the short term, to neurotransmitters, upregulation of CD3zeta levels by even brief exposure to neurotransmitters can indicate the increase in disease-fighting capabilities. Thus, monitoring CD3 zeta levels of treated T-cells can both provide a method of assessing the effectiveness of treatment of the T-cells, and provide an important parameter for determining course of treatment, dosage and expected treatment outcome.

### Example 2

### Blocking Ion Channels in Normal Human T-Cells Triggers Robust TNFα Secretion

As detailed hereinabove, secretion of TNFα is an important component of the T-cell response to activating stimuli, and likely facilitates T-cell extravasation across tissue barriers. TNFα elevation induced by ion channel block may have important clinical implications, as boosting TNFα in certain diseases (e.g. cancer and immunodeficiencies) and arresting it in others (e.g. Rheumatoid Arthritis, Crohn's and other autoimmune/inflammatory diseases) is a major therapeutic goal. In order to clarify the role of ion channels in human T-cell TNFα secretion, and to assess the effects of specific ion channel blockers on T-cell activation, human T-cells were exposed to a variety of ion channel blockers ex-vivo, and levels of TNFα secretion were determined.

Materials and Experimental Method

Ion channel blockers: Specified for each ion channel blocker used herein are its full name, its abbreviation and manufacturer in brackets, and its effective concentrations (derived from the respective manufacturer's data sheets and/or the literature). Further detailed information can be found at the International Union of Pharmacology website. The blockers included: 4-Aminopyridine (4-AP, Sigma, St. Louis, Mo.), 10 μM-1 mM; Tetraethylammonium (TEA, Sigma), 100 μM-10 mM; Quinine (Sigma), 1-10 μM; Clotrimazole (CLT, Agis Industries, Bnei Brak, Israel), 10 nM-10 μM; rCharybdotoxin (CTX, Alomone labs, Jerusalem, Israel), 10-100 nM; Kaliotoxin (KTX, Alomone), 1-100 nM; rMargatoxin (MgTX, Alomone), 50 pM-50 nM; Dendrotoxin-K (DTX-K, Alomone), 10-100 nM; Tetrodotoxin (TTX, Alomone), 100 nM-1 μM; NPPB (Tocris Cookson, Avonmouth, UK), 100-200 μM; R-(+)-Bay K8644 (+Bay K, Tocris) 10 nM-1 μM; CNQX (Tocris), 100 nM-50 μM.

Human T-cells: Normal human T-cells were purified from peripheral blood of healthy donors as described (19, 23, 61), and the resulting cell population, containing ≧90% T-cells, was suspended in RPMI medium supplemented with 10% FCS, 1% LGlutamine and antibiotics (Biological Industries, Bet Haemek, Israel) and maintained at 2×10₆ cells/ml (37° C./5% CO₂).

Determination of TNFα, IFNγ, IL-10 and IL-4 by ELISA: Freshly-purified normal resting human T-cells (2×10⁶/ml) were incubated in 24-well plates (Costar, Corning, N.Y.) and the various channel blockers were added. Importantly, no other stimulating molecule (e.g. phorbol esters, antigens, anti-CD3/CD28 mAbs) was added. Cytokine 23 levels were measured in supernatants after 24 hr for TNFα and IFNγ and after 72 hr for IL-10 and IL-4, by quantitative sandwich ELISA (R&D, Minneapolis, Minn.), according to the manufacturer's instructions.

Results

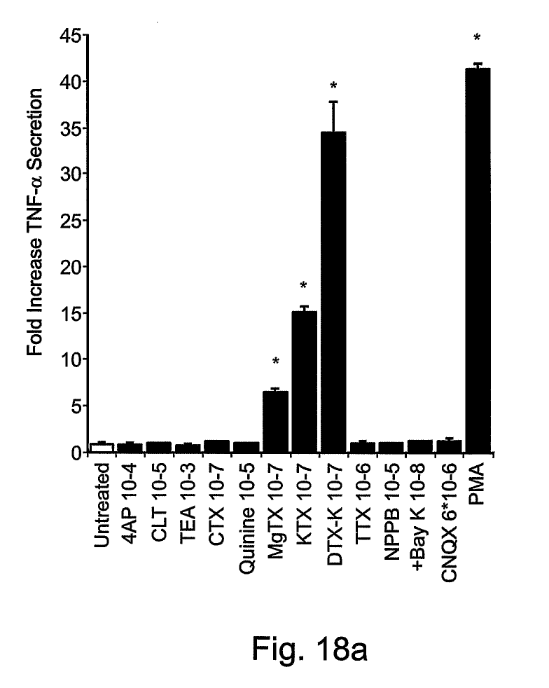

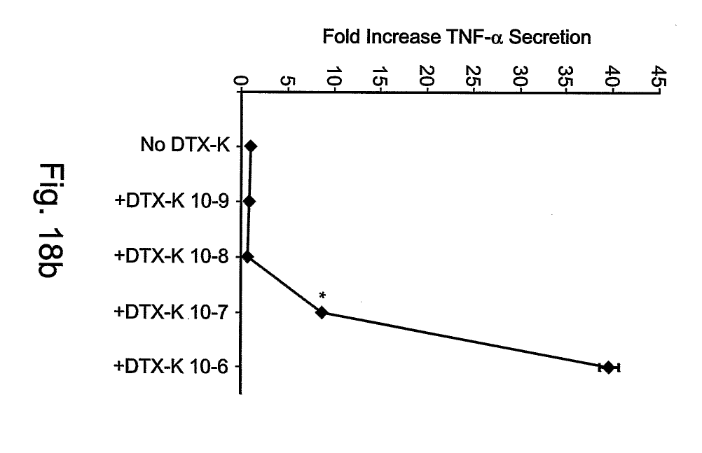

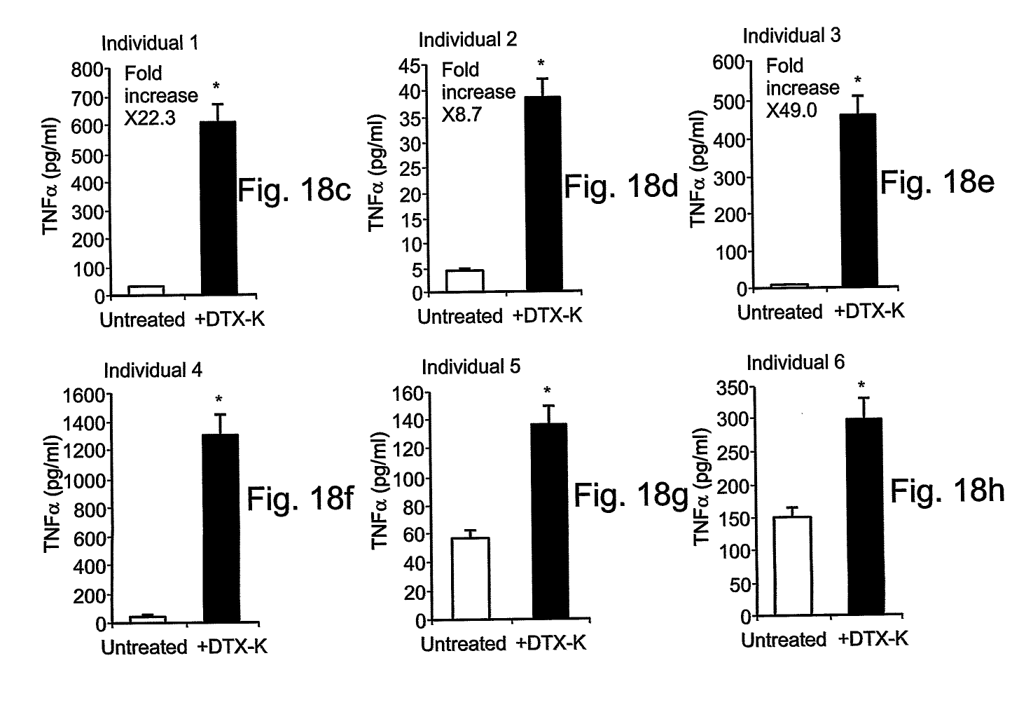

12 specific ion channel blockers were tested to determine their effect on TNFα secretion in “resting” normal human T-cells. FIG. 18a demonstrates the effective triggering of marked TNFα secretion by 100 nM dendrotoxin-K, a selective Kv1.1 blocker, and somewhat weaker effect of quinine and kaliotoxin (KTX). The triggering effect of DTX-K was dose dependent, as shown in FIG. 18b. FIG. 18c-h, show that despite the different background levels of T-cell derived TNFα, DTX-K (24 hr, 100 nM) triggered marked TNFα secretion by T-cells from six different individuals, indicating the universality of the effect. Of note, while “classical” T-cell activation by PMA resulted in increased secretion of TNFα, IFNγ, IL-4 and IL-10, ion channel block by DTX-K selectively increased only TNFα secretion in the treated T-cells (data not shown).

DTX-K had no significant effect on cell survival or proliferation of the human T-cells (data not shown).

Thus, these results indicate that particular highly selective ion channel blockers can on their own (i.e. in the absence of any additional stimuli) trigger robust and exclusive TNFα secretion in normal, “resting” human T-cells. Therefore, such particular ion channel blockers can be used to “boost” T-cells in the methods and devices of the present invention, Further, monitoring TNFα secretion in treated T-cells can be an effective means for assessing the T-cell response to boosting by the methods of the present invention.

While the invention has been described with respect to several preferred embodiments, it will be appreciated that these are set forth merely for purposes of example, and that many other variations, modifications and applications of the invention may be made.

1. A method of augmenting the ability of certain body cells of a patient for fighting disease in the patient, comprising:
exposing, for a predetermined exposure period, a first substance of cells having the ability of fighting disease to a second substance having a cell-boosting capability to augment the ability of the cells to fight the disease;
characterized in that said first substance is exposed to said second substance by mixing the two substances in a mixing device providing hermetically-sealed conditions effective to maintain sterility in both substances during the mixing thereof and for said predetermined exposure period.

2. The method of claim 1, wherein said cells of said first substance are T-cells.

3-13. (canceled)

14. The method of claim 1, wherein said first substance of cells is mixed with said second substance by:
including said second substance in a dispensing port of a mixing device, said drug dispensing port having a first compartment, said first compartment having first and second needle-pierceable walls on its opposite sides;
including said first substance of cells in fluid communication with a second compartment of the drug dispensing port located on one side of said first compartment to face said first needle-pierceable wall thereof,
wherein said second compartment being movable from a normal position to an actuated position with respect to said first compartment and carrying two hollow needles facing said first needle-pierceable wall but spaced there from in said normal position of the second compartment, the first of said needles having a length to pierce both walls of said first compartment in the actuated position of the second compartment, and the second of said needles having a length to pierce said first, but not said second wall of said first compartment in the actuated position of the second compartment;
providing a mixing compartment in said mixing device located on the opposite side of said first compartment to face said second needle-pierceable wall thereof;
and moving said second compartment to its actuated position to cause said first hollow needle to pierce both of said needle-pierceable walls of said first compartment and said second hollow needle to pierce said first wall of said first compartment;
said first hollow needle having a pointed open end and a side opening spaced from said pointed open end such that, in the actuated position of the second compartment, said side opening is located within said first compartment, and said pointed open end is located in said third compartment;
said second hollow needle having a pointed open end spaced so as to be located within said first compartment in the actuated position of the second compartment, and an upper opening spaced so as to be in fluid communication with said first substance of cells located within said second compartment in the actuated position of the second compartment.

15. The method of claim 14, wherein said mixing compartment is a cell chamber for mixing said first substance of cells and said second substance for said predetermined exposure period.

16. The method of claim 14, wherein said mixing device comprises a receptacle having a plurality of said mixing compartments.

17. The method of claim 1, wherein said first substance of cells is mixed with said second substance by:
including one of said substances in a first compartment of a mixing device, said first compartment having first and second needle-pierceable walls on its opposite sides;
including the other of said substances in a second compartment of the mixing device located on one side of said first compartment to face said first needle-pierceable wall thereof, said second compartment being movable from a normal position to an actuated position with respect to said first compartment and carrying two hollow needles facing said first needle-pierceable wall but spaced there from in said normal position of the second compartment, said needles having a length to pierce both walls in the actuated position of the second compartment;
providing a third compartment in said mixing device located on the opposite side of said first compartment to face said second needle-pierceable wall thereof;
and moving said second compartment to its actuated position to cause both hollow needles to pierce both of said needle-pierceable walls of said first compartment;
one of said hollow needles having a pointed closed end, a first side opening spaced from said pointed closed end so as to be located within said first compartment in the actuated position of the second compartment, and a second side opening spaced from said pointed closed end so as to be located within said second compartment in the actuated position of the second compartment;
the other of said hollow needles having a pointed open end and a side opening spaced from said pointed open end such that, in the actuated position of the second compartment, said side opening is located within said second compartment, and said pointed open end is located in said third compartment.

18. The method of claim 17, wherein said third compartment includes an outlet normally closed by a valve but openable after said predetermined exposure period to outlet the cells of augmented disease-fighting ability.

19. The method of claim 1, wherein said first substance is mixed with said second substance by:
including said second substance having the cell-boosting capability in a compartment having an injection port and an outlet port; and injecting said first substance of cells into said compartment through said injection port.

20. (canceled)

21. The method of claim 19, wherein said second substance having the cell-boosting capability is included in each of a plurality of compartments interconnected in series by one-way valves; each of said compartments including a said injection port such that the quantity of said second substance having cell-boosting capability to be exposed to said first substance of cells may be selected as desired by injecting said first substance of cells into the injection port of a selected compartment containing said second substance of cell-boosting capability.

22. The method of claim 1, wherein said first substance of cells is mixed with said second substance of cell-boosting capability by:
introducing said second substance in capsule form into a compartment;
rupturing said capsule by the application of pressure thereto; and
injecting said first substance into said compartment to thereby mix with said second substance.

23. The method of claim 22, wherein:
said second substance is provided in a plurality of capsules each introduced into one of a plurality of compartments;
a capsule in one or more selected compartments is ruptured;
and said first substance of cells is injected into each compartment where a capsule is ruptured to thereby enable augmenting the disease-fighting ability of a desired selected quantity of the separated cells of said first substance.

24. The method of claim 23, wherein said plurality of compartments are included in a plastic bag having internal partitions dividing the interior of the plastic bag into a plurality of compartments each having a capsule containing said second substance.

25. The method according to claim 24, wherein said plurality of compartments are arranged in a series, with each compartment, except for the last one in the series, having an outlet port communicating with the interior of the next compartment in the series via a one-way valve.

26. The method according to claim 25, wherein said plurality of compartments are arranged in a series; and wherein each compartment has, or is marked to indicate, an inlet opening at one end of the compartment, and an outlet opening at the opposite end of the compartment.

27. A mixing device for use in the method of claim 1 for mixing two substances in a hermetically-sealed manner, said mixing device comprising:
a first compartment for one of said substances, said first compartment having first and second needle-pierceable walls on its opposite sides;
a second compartment for the other of said substances located on one side of said first compartment facing said first needle-pierceable wall thereof and movable from a normal position to an actuated position with respect to said first compartment;
two hollow needles carried by said second compartment facing said first needle-pierceable wall thereof and of a length to pierce both walls in the actuated position of the second compartment;
and a third compartment located on the opposite side of said first compartment facing said second needle-pierceable wall thereof;
and moving said second compartment to its actuated position to cause both hollow needles to pierce both of said needle-pierceable walls of said first compartment;
one of said hollow needles having a pointed closed end, a first side opening spaced from said pointed closed end so as to be located within said first compartment in the actuated position of the second compartment, and a second side opening spaced from said pointed closed end so as to be located within said second compartment in the actuated position of the second compartment.

28. The mixing device of claim 27, wherein said third compartment includes an outlet normally closed by a valve but openable after a predetermined exposure period to outlet the mixed substances.

29. A mixing device for use in the method of claim 1 for mixing two substances in a hermetically-sealed manner, said mixing device comprising:
a receptacle having a plurality of compartments arranged in a series, each for receiving a quantity of one of said substances;
each of said compartments having an outlet port; the outlet port of each compartment except for the last in the series being connected to the interior of the next compartment in the series via a one-way valve permitting flow only in the direction to the interior of the next compartment in the series;
each of said compartments being formed with, or marked for, an inlet port for introducing the other of said substances, such that the quantity of said one substance to be mixed with said other substance may be selected by introducing said other substance into the inlet port of a selected compartment in the series.

30. A mixing device for use in the method of claim 1, for mixing two substances in a hermetically sealed manner, the mixing device comprising:
a receptacle having a plurality of mixing compartments arranged in a series, each for receiving a quantity of one of said substances;
each of said compartments having a drug dispensing port, a fluid inlet port and an outlet port, said outlet port of each compartment being connected to a one way valve permitting flow of said mixed substances into an outlet pipe;
wherein said drug dispensing port comprises:
a first compartment for said second substance, said first compartment having first and second needle-pierceable walls on its opposite sides;
a second compartment in fluid communication with said first substance of cells located above said first compartment facing said first needle-pierceable wall thereof and movable from a normal position to an actuated position with respect to said first compartment;
a first and a second hollow needle carried by said second compartment facing said first needle-pierceable wall thereof, said first hollow needle of a length to pierce both walls in the actuated position of the second compartment, and said second hollow needle of a length to pierce said first but not said second walls in the actuated position of the second compartment;
wherein said mixing compartment is located on the opposite side of said first compartment facing said second needle-pierceable wall thereof;
and moving said second compartment to its actuated position causes both hollow needles to pierce said first needle-pierceable wall of said first compartment and said first hollow needle to pierce both said needle pierceable walls of said first compartment;
wherein said first hollow needle has a pointed open end, a first side opening spaced from said pointed open end so as to be located within said first compartment in the actuated position of the second compartment, said pointed open end spaced to be located in said mixing compartment in the actuated position of the second compartment;
said second hollow needle having a pointed open end spaced so as to be located within said first compartment in the actuated position of the second compartment, and an upper opening spaced so as to be in fluid communication with said first substance of cells located within said second compartment in the actuated position of the second compartment.

31. The mixing device of claim 30, wherein said mixing compartments are separated by a non-removable separator.

32. The mixing device of claim 30, wherein said mixing compartments are separated by a removable separator.

33. A mixing device for use in the method of claim 1 for mixing two substances in a hermetically-sealed manner, said mixing device comprising:
a receptacle having a plurality of compartments each for introducing a quantity of one of said substances;
each of said compartments including a capsule containing a quantity of the other of said substances;
each of the capsules being rupturable by pressure to release the substance therein for exposure to the substance introduced into the respective compartment, thereby enabling a selection to be made, according to the number of capsules ruptured and the number of compartments into which said first substance is introduced, as to the quantity of the two substances to be mixed.

34. The mixing device of claim 33, wherein said plurality of compartments are included in a plastic bag having internal partitions dividing the interior of the plastic bag into a plurality of compartments each having a capsule containing said second substance.

35. The mixing device of claim 34, wherein said plurality of compartments are arranged in a series, with each compartment, except for the last in the series, having an outlet port communicating with the interior of the next compartment in the series via a one-way valve.

36. The mixing device of claim 34, wherein said plurality of compartments are arranged in a series; and wherein each compartment has, or. is marked to indicate, an inlet opening at one end of the compartment, and an outlet opening at the opposite end of the compartment.

37-40. (canceled)
